(19) United States
(12) Patent Application Publication
(10) Pub. No.: US 2023/0358649 A1
(43) Pub. Date: Nov. 9, 2023

(54) COMPOSITIONS AND METHODS FOR REMOVAL OF BOUND DETECTION AGENTS

(71) Applicant: VECTOR LABORATORIES, INC., Newark, CA (US)

(72) Inventors: August ESTABROOK, Castro Valley, CA (US); Dennis HICKS, Pittsburg, CA (US); Pamela JAMES, Redwood City, CA (US); Timothy KARPISHIN, San Mateo, CA (US); Erika LEONARD, Castro Valley, CA (US); Shamali Roy CHOUDHURY, San Ramon, CA (US)

(21) Appl. No.: 18/313,997

(22) Filed: May 8, 2023

Related U.S. Application Data
(60) Provisional application No. 63/339,703, filed on May 9, 2022.

(51) Int. Cl. G01N 33/58; C12Q 1/6804; G01N 1/34; G01N 21/64

(52) U.S. Cl. CPC G01N 1/34; C12Q 1/6804; G01N 21/6428; G01N 33/582; G01N 2021/6441; G01N 2458/10

(57) ABSTRACT

Disclosed are compositions, methods, systems, and kits for the detection of markers of interest, which may allow for sequential multiplexing. In some cases, the methods comprise sequential staining of biological samples with detection sets and removal of bound detection agents which may allow for sequential staining with additional detection agents.

### CROSS REFERENCE TO RELATED APPLICATION

The present application claims the benefit of priority under 35 U.S.C. § 119(e) to U.S. Provisional Application No. 63/339,703, COMPOSITIONS AND METHODS FOR REMOVAL OF BOUND DETECTION AGENTS, filed on May 9, 2022, which is incorporated by reference in its entirety.

### FIELD

Described herein are compositions and methods for removal of bound detection agents.

### BACKGROUND

Various techniques related to staining with detection agents are widely used in both clinical diagnostic applications and laboratory research. For example, clinically, immunostaining can be used in histopathology for the diagnosis of specific types of disease based on the detection of associated biomarkers. In laboratory science, immunostaining can be used for a variety of applications based on investigating the presence or absence of a protein, its tissue distribution, its sub-cellular localization, and of changes in protein expression or degradation. However, these techniques may be limited by the number of biomarkers that can be detected in a single sample.

Multiplex techniques have emerged that allow for simultaneous detection of multiple markers on a single sample or sequential detection of one or more markers on a single sample. Simultaneous detection is limited to the number of uniquely detectable labels available, and is also limited by the cross-reactivity that occurs between detection agents in various workflows. Sequential detection can be limited by harsh stripping processes that can contribute to biomarker and/or specimen degradation.

There is a need for compositions and techniques, i.e. techniques using such compositions, that are capable of removing detection agents from a surface, without negatively impacting the surface. There is also a need for compositions and techniques, i.e. techniques using such compositions, that are allow for subsequent and/or multiple cycles of staining, detection, and detection agent removal. For example, removal of bound detection agents from a tissue without degrading the tissue can allow for subsequent staining and detection cycles, as well as multiplex techniques. Currently available products do not allow for such processes.

### SUMMARY

Various embodiments described herein can provide detection agent removal reagent compositions and methods of using the same.

Embodiments of the present disclosure include compositions and methods for removing one or more bound detection agent from a stained biological sample or surface, where the composition includes a reductant and a denaturant.

Embodiments of the disclosure also relate to methods for removing one or more bound detection agent from a stained biological sample or surface, the method including: (i) providing a stained biological sample or surface; (ii) applying a reductant to the sample; and (iii) applying a denaturant to the sample.

In some embodiments, a sequential method for multiplex detection is described herein. Sequential methods in accordance with the present invention can include: (i) identifying a plurality of target markers of interest in a biological sample or a surface; (ii) staining the biological sample or surface with a first plurality of detection agents specific for a first plurality of target markers of interest; (iii) optionally, detecting a visual signal that indicates the presence or absence of the markers of interest in step (ii); (iv) removing the first plurality of bound detection agents from the stained biological sample or surface; (v) staining the biological sample or surface with a second plurality of detection agents specific for a second plurality of target markers of interest; (vi) optionally, detecting a visual signal that indicates the presence or absence of the markers of interest of step (v); and (vii) optionally, repeating steps (iv)-(vi) for detection of additional markers of interest. Sequential methods in accordance with the present invention can also include: (i) identifying a plurality of target markers of interest in a biological sample or a surface; (ii) staining the biological sample or surface with a first plurality of detection agents specific for a first plurality of target markers of interest; (iii) removing the first plurality of bound detection agents from the stained biological sample or surface; (iv) staining the biological sample or surface with a second plurality of detection agents specific for a second plurality of target markers of interest; (v) optionally, repeating steps (iii)-(iv) for detection of additional markers of interest; and (vi) detecting a visual signal that indicates the presence or absence of the markers of interest. In some embodiments of the methods, steps (ii)-(vi) are repeated 1, 2, 3, 4, 5, 6, 7, 8, 9, or 10 times. In some embodiments of the methods, steps (ii)-(iv) are repeated 1, 2, 3, 4, 5, 6, 7, 8, 9, or 10 times.

In some embodiments, the plurality of target markers of interest includes at least 1, 2, 3, 4, 5, 6, 7, 8, 9, 10, 15, 20, 25, 20, 35, 40, 45, or 50 markers of interest.

In some embodiments of the compositions and methods, the biological sample may be a tissue sample. In some embodiments, the tissue includes lung, liver, heart, colon, nervous system, skin, lymph gland, musculoskeletal, smooth muscle, spleen, eye, sinus, nasal mucosa, larynx, gastrointestinal tract tissue, reproductive organ, breast, prostate, salivary gland, tonsil, connective tissue, epithelium, cardiovascular, or kidney tissue.

In some embodiments of the compositions and methods, the surface may be a sensor, an electrode, and/or a hydrogel. In some embodiments of the compositions and methods, the sensor and/or electrode includes one or more type of metal. In some embodiments of the compositions and methods, the sensor can be functionalized with one or more binding agent and/or reactive group. In some embodiments of the compositions and methods, the binding agent includes a protein and/or a hydrogel. In some embodiments of the compositions and methods, the sensor is functionalized with a hydrogel.

In some embodiments of the compositions and methods, the reductant includes at least one of beta-mercaptoethanol, Lithium aluminum hydride (LiAlH4), Red-Al (NaAlH2(OCH2CH2OCH3)2), Nascent (atomic) hydrogen, Hydrogen without or with a suitable catalyst e.g. a Lindlar catalyst, Sodium amalgam (Na(Hg)), Sodium-lead alloy (Na + Pb), Zinc amalgam (Zn(Hg)), Diborane Sodium borohydride (NaBH4), Compounds containing the Fe2+ ion, such as iron(II) sulfate, Compounds containing the Sn2+ ion such as tin(II) chloride, Sulfur dioxide, Sulfite compounds, Dithionates (Na2S2O6), Thiosulfates (Na2S2O3), Iodides (KI), Hydrogen peroxide (H2O2), Hydrazine (Wolff-Kishner reduction), Diisobutylaluminium hydride (DIBAL-H), Oxalic acid (C2H2O4), Formic acid (HCOOH), Ascorbic acid (C6H8O6), Reducing sugars, Phosphites, hypophosphites, and phosphorous acid, Dithiothreitol (DTT), Carbon monoxide (CO), Cyanides in hydrochemical metallurgical processes, Carbon (C), Tris-2-carboxyethylphosphine hydrochloride (TCEP), and/or tris-hydroxypropyl phosphine (THPP). In some embodiments, the reductant includes tris-hydroxypropyl phosphine.

In some embodiments, the denaturant includes at least one of guanidinium thiocyanate, ammonium thiocyanate, urea, guanidine, guanidium chloride, lithium perchlorate, sodium dodecyl sulfate, acetic acid, trichloroacetic acid, sulfosalicylic acid, sodium bicarbonate, Dodecaborate (B₁₂H₁₂)⁻², ethanol, formaldehyde, and/or glutaraldehyde. In some embodiments, the denaturant includes guanidinium thiocyanate.

In some embodiments of the compositions and methods, the reductant includes tris-hydroxypropyl phosphine, and the denaturant includes guanidinium thiocyanate. In some embodiments, the reductant includes THPP in aqueous solution at a concentration of between about 1 to 100 mg/mL, and the denaturant includes guanidinium thiocyanate in aqueous solution at a concentration of between about 1 M to 6 M.

In some embodiments of the compositions and methods, the reductant and denaturant are separate solutions. In some embodiments of the compositions and methods, the reductant and denaturant are together in solution. In some embodiments of the compositions and methods, the composition includes one or more aqueous solution.

In some embodiments of the compositions and methods, the one or more bound detection agent can include at least one of a dye, antibody, nanobody, lectin, glycan-binding protein, recombinant protein, peptide, antibody fragment such as Fab, streptavidin, avidin, dendrimer, aptamer, enzyme such as horse radish peroxidase, alkaline phosphatase, or glucose oxidase, oligonucleotide, optionally labeled probe such as an oligonucleotide probe, single-stranded DNA probe, double-stranded DNA probe, branched DNA probe, and/or RNA probe, and/or any combination thereof. In some embodiments of the compositions and methods, the bound detection agent includes one or more primary and/or secondary antibody. In some embodiments of the compositions and methods, the bound detection agent can be labelled or unlabeled. In some embodiments of the compositions and methods, the label on the bound detection agent can include a dye, an enzyme, a mass tag, a nanoparticle, a quantum dot, or an oligonucleotide.

In some embodiments, the methods can be performed at room temperature. In some embodiments, the compositions can be stored at room temperature, and/or at refrigeration temperature.

In some embodiments of the compositions and methods, at least 60%, 70%, 75%, 80%, 85%, 90%, 91%, 92%, 93%, 94%, 95%, 96%, 97%, 98%, 99%, or more of the total quantity of the one or more bound detection agent can be removed from the stained biological sample or surface. In some embodiments of the compositions and methods, substantially all of the total quantity of the one or more bound detection agent can be removed from the stained sample or surface.

In some embodiments of the compositions and methods, the reductant and the denaturant can be applied simultaneously. In some embodiments of the compositions and methods, the reductant can be applied to the stained sample prior to application of the denaturant. In some embodiments of the compositions and methods, the denaturant can be applied to the stained sample prior to application of reductant.

In some embodiments, the methods further include one or more staining steps.

In some embodiments, the methods further include a second, third, fourth, fifth, sixth, or seventh detection agent removal step.

In some embodiments of the methods, the tissue integrity and/or morphology and/or nuclear features and/or antigenicity can be preserved after the first, second, third, fourth, fifth, sixth, and/or seventh detection agent removal steps.

In some embodiments of the methods, the process of detecting a visual signal includes capturing an image of the stained biological sample or surface. In some embodiments of the methods, the captured images from each sequential round of staining can be compiled into one multiplexed image.

In some cases, a kit for removing a bound detection agent from a stained biological sample is described herein. The kit can include a reductant and a denaturant, for example, as in the compositions as described herein.

In some cases, a system for the detection of a target marker of interest is described herein. The system includes (i) a station for providing a sample; (ii) a station for staining at least one biomarker of interest in the sample using a detection agent; (iii) a station for detecting the target marker of interest; (iv) a station to analyze the results to determine the presence or amount of the one or more biomarkers in the sample; and (v) a station for removing the detection agent.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 depicts images of a tissue following incubation with Reagent 1 (Tris-hydroxy propyl phosphene) and Reagent 2 (ammonium thiocyanate) at 8 M, 4 M, and 2 M according to Example 2 described herein.

FIG. 2 depicts images of a tissue following incubation with Reagent 1 (Tris-hydroxy propyl phosphene) and Reagent 2 (guanidine thiocyanate): supernatant of saturated Reagent 2, 50% saturation Reagent 2, or 25% saturation Reagent 2 according to Example 3 described herein.

FIG. 3 depicts images of a tissue following incubation with Reagent 1 (Tris-hydroxy propyl phosphene) and Reagent 2 (sodium perchlorate): supernatant of saturated Reagent 2, 50% saturation Reagent 2, or 25% saturation Reagent 2 according to Example 4 described herein.

FIG. 4 depicts examples of autofluorescence, using anti-Ki67p + Akoya Opal 570 tyramide + MWT + Akoya Opal 520 tyramide. The panel on the left shows positive staining viewed in the red channel, and the panel on the right shows the chase with no staining viewed in the green channel.

FIGS. 5A-5D depicts complete stripping using both reagents, showing anti-AE1/AE3 staining on tonsil tissue at 20x (FIGS. 5A and 5B) and anti-S100p staining on colon tissue at 20× (FIGS. 5C and 5D). Both samples show positive staining before DARR treatment, and complete stripping using both of the detection agent removal reagents (DARR) (FIGS. 5A and 5C), and incomplete stripping using only one reagent or the other (FIGS. 5B and 5D).

FIGS. 6A-6D depicts DARR removal of detection reagents. Tests were run on colon tissue and show results before (top panels) and after DARR treatment (bottom panels). Tests were run on AE1/AE3 (FIG. 6A), CD20 (FIG. 6B), Erlin-2 (FIG. 6D), Ki67p (FIG. 6D), and S100p (FIG. 6E) in FFPE colon tissue.

FIGS. 7A-7B depicts the verification of complete removal of antibodies using tyramide signal amplification (TSA). Anti-CD8 (FIG. 7A) and anti-Ki67p (FIG. 7B) are both stripped well by both DARR and microwave treatment (MWT).

FIG. 8 depicts the minimal impact on Opal dye intensity with no stripping, 6 cycles of DARR stripping, and 6 cycles of MWT stripping.

FIGS. 9A-9D shows DARR vs Biocare stripping performance in the TSA workflow. Anti-Ki67p and anti-AE1/AE3 are both stripped well by DARR (FIG. 9A and FIG. 9C, respectively), but stripping with the Biocare reagent results in considerable residual staining demonstrating the primary antibody used for staining was not completely removed by the Biocare reagent. (FIGS. 9B and 9D, respectively).

FIGS. 10A-10D shows the DARR vs Biocare Denaturing Solution stripping performance in the IF workflow. Anti-AE1/AE3 and anti-S100p are both stripped well by DARR (FIG. 10A and FIG. 10C, respectively), but stripping with the Biocare Denaturing Solution results in considerable residual staining demonstrating the primary antibody used for staining was not completely removed by the Biocare Denaturing Solution. (FIGS. 10B and 10D, respectively).

FIGS. 11A-11D shows the assessment of antigenicity after DARR and MWT in the TSA workflow. There is no loss of antigenicity after 6 cycles of DARR using anti-PDL-1 (FIG. 11A), anti-CD3 (FIG. 11B), anti-Ki67p (FIG. 11C), or anti-CD20 (FIG. 11D) staining.

FIGS. 12A-12H shows the assessment of antigenicity after DARR in the IF workflow. There is no loss of antigenicity after 1 or 6 cycles of DARR using anti-CA19-9 in colon tissue (FIG. 12A), anti-CD3 in tonsil tissue (FIG. 12B), anti-CD11, anti-SMA, anti-PAP, and anti-PSA in prostate tissue (FIG. 12C, FIG. 12D, FIG. 12E, and FIG. 12F, respectively), anti-GFAP in brain tissue (FIG. 12G), and anti-C11 in colon carcinoma (FIG. 12H).

FIGS. 13A-13D shows the tissue damage, or lack thereof, after 6 cycles of pressure cooking and DARR. DARR was found to preserve tissue integrity in breast cancer tissue (FIG. 13A, 4× magnification), colon cancer tissue (FIG. 13B, 4× magnification), tonsil tissue (FIG. 13C,10× magnification; FIG. 13D, 20× magnification; FIG. 13E, 20× magnification), whereas pressure cooker stripping results in a complete loss of the fatty matrix in breast cancer tissue, a significant loss of connective tissue matrix in colon cancer tissue, and general tissue damage in tonsil tissue with 6 rounds of stripping.

FIGS. 14A-14B shows the tissue damage, or lack thereof, after 6 cycles of MWT and DARR. DARR was found to preserve tissue integrity in FFPE skin tissue (FIG. 14A) and breast carcinoma (FIG. 14B), whereas MWT results in notable damage to tissue morphology.

FIGS. 15A-15B shows the result of a 6 stripping cycle multiplex workflow. FIG. 15A and FIG. 15B show imaging from different combinations of 6 primary binding agents and one DAPI staining, which can be aligned, merged and visualized after performing DARR treatment between each round of staining.

FIGS. 16A-16F shows the results of reversing the order of DARR components. FFPE tonsil and colon tissues stained with anti-AE1/AE3, anti-Ki67p, and anti-S100p and DARR performed in the original order (FIG. 16A, FIG. 16C, and FIG. 16E, respectively), and with anti-AE1/AE3, anti-Ki67p, and anti-S100p staining followed by DARR performed in the reverse order (FIG. 16B, FIG. 16D, and FIG. 16F, respectively).

FIG. 17 shows the method of using DARR for detecting analytes such as proteins and nucleic acids using chemistries in a sequential manner over a certain number of cycles with imaging at each step or after all the cycles are completed.

### DETAILED DESCRIPTION OF THE INVENTION

Embodiments described herein relate generally to detection agent removal reagent compositions. In some cases, the present invention provides compositions, methods, kits, and systems for the removal of bound detection agents that may allow for sequential multiplex staining. In some embodiments, the present invention provides compositions, methods, kits, and systems for the removal of bound detection agents from a surface.

It should be understood that, unless clearly indicated to the contrary, in any methods claimed herein that include more than one step or act, the order of the steps or acts of the method is not necessarily limited to the order in which the steps or acts of the method are recited.

### Definitions and Descriptions

The terms “invention,” “the invention,” “the present invention,” “embodiment,” “certain embodiment” and the like are used herein and are intended to refer broadly to all the subject matter of this patent application and the claims below. Statements containing these terms should be understood not to limit the subject matter described herein or to limit the meaning or scope of the patent claims below.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of”, or “exactly one of”, or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In various embodiments, the term “about” indicates the designated value ± up to 10%, up to ± 5%, or up to ± 1%.

As used herein, the term “biological sample” or “tissue sample” refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the terms “biomarker” or “marker” generally refer to any measurable substance present on a sample. In some embodiments, the term “marker” or “biomarker” refers to a substance detectable on the surface of a sample, such as, for example, a label attached to a sample, or attached to component of a sample. In some embodiments, the term “marker” or “biomarker” refers to a measurable indicator of some biological state or condition. For example, the marker or biomarker can be from a subject, where the presence of the marker or biomarker is indicative of some phenomenon. Non-limiting examples of such phenomenon can include a disease state, a condition, or exposure to a compound or environmental condition. In various embodiments described herein, markers or biomarkers may be used for diagnostic purposes (e.g., to diagnose a health state, a disease state). The term “biomarker” can be used interchangeably with the term “marker.” In particular, a biomarker may be a protein or peptide, e.g., a protein, that can be specifically stained, and which is indicative of a biological feature of the cell, e.g., the cell type or the physiological state of the cell. An immune cell marker is a biomarker that is selectively indicative of a feature that relates to an immune response of a mammal. A biomarker may be used to determine how well the body responds to a treatment for a disease or condition or if the subject is predisposed to a disease or condition. In the context of cancer, a biomarker refers to a biological substance that is indicative of the presence of cancer in the body. A biomarker may be a molecule secreted by a tumor or a specific response of the body to the presence of cancer. Genetic, epigenetic, proteomic, glycomic, and imaging biomarkers can be used for cancer diagnosis, prognosis, and epidemiology. Such biomarkers can be assayed in non-invasively collected biofluids like blood or serum. A detection reagent is a marker is used to stain one or more species on a sample. For example, a detection reagent can be a compound used to stain sample proteins, glycans, lectins, and the like.

The term “analyte” in the context of the present teachings can mean any substance to be measured, and can encompass biomarkers, markers, nucleic acids, electrolytes, metabolites, proteins, sugars, carbohydrates, fats, lipids, cytokines, chemokines, growth factors, proteins, peptides, nucleic acids, oligonucleotides, metabolites, mutations, variants, polymorphisms, modifications, fragments, subunits, degradation products and other elements.

To “analyze” includes determining a value or set of values associated with a sample by measurement of analyte levels in the sample. “Analyze” may further comprise comparing the levels against constituent levels in a sample or set of samples from the same subject or other subject(s). The markers of the present teachings can be analyzed by any of various conventional methods known in the art. Some such methods include, but are not limited to, measuring serum protein or sugar or metabolite or other analyte level, measuring enzymatic activity, and measuring gene expression.

The term “antibody” refers to any immunoglobulin-like molecule that reversibly binds to another protein, nucleic acid, carbohydrate, or any analyte with the desired selectivity. Thus, the term includes any such molecule that is capable of selectively binding to a marker of the present teachings. The term includes an immunoglobulin molecule capable of binding an epitope present on an antigen. The term is intended to encompass not only intact immunoglobulin molecules, such as monoclonal and polyclonal antibodies, but also antibody isotypes, recombinant antibodies, bi-specific antibodies, humanized antibodies, chimeric antibodies, anti-idiopathic (anti-ID) antibodies, single-chain antibodies, Fab fragments, F(ab′) fragments, F(ab′)₂, fusion protein antibody fragments, immunoglobulin fragments, Fᵥ fragments, single chain Fᵥ fragments, and chimeras comprising an immunoglobulin sequence and any modifications of the foregoing that comprise an antigen recognition site of the desired selectivity.

An “immunoassay” as used herein refers to a biochemical assay that uses one or more antibodies to measure the presence or concentration of an analyte or marker in a biological sample. A “multiplex immunoassay” as used herein refers to a biochemical assay that uses more than one antibody to simultaneously, or through sequential binding and, optionally, stripping of the immunoglobulin, measure multiple analytes.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival cervicular fluid, bone marrow, cerebrospinal fluid (CSF), saliva, mucous, sputum, semen, sweat, urine, or any other bodily fluids. “Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art.

The term “staining” or “stained” as used herein refers to the detection of specific markers of interest in a biological sample using a detection agent (e.g., antibody, dye, hybridization probe, or fluorochrome). Staining may refer to any method that identifies a marker of interest by creating a detectable (readable) signal. Staining may include immunostaining. Immunostaining is the use of antibodies to detect proteins in a sample. Staining may also include histochemical staining. Histochemical staining is the use of stains to detect chemical constitution of cells and tissues, including mucins, lipids, nucleic acids, amyloid, microorganisms, and other proteins. Staining may further include in situ hybridization (ISH). ISH is a technique used to localize a sequence of DNA or RNA in a biological sample using hybridization probes.

The term “autofluorescence” refers to a signal that comes from the sample (e.g. a tissue) itself intrinsically or as a result of the fixative used and is not associated with staining (IF, IHC, TSA, etc.). For example, autofluorescence in tissue sections can be due to aldehyde fixation, the presence of red blood cells, and structural elements, such as collagen and elastin. Autofluorescence can be present in FFPE tissues and can manifest as, for example, a spot that is visible in several different channels and is present in specific areas, and/or a general background glow from the tissue. Proper control sections can be used to identify autofluorescent elements in tissues.

The term “room temperature” as used herein refers to a temperature of from about 15° C. to about 30° C.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. Additionally, any reference referred to as being “incorporated herein” is to be understood as being incorporated in its entirety.

### Detection Agent Removal Reagent Compositions

Various formulations have been proposed to date for the evaluation of antibody/antigen interactions and methods of stripping antibodies and other reagents from various surfaces, including the removal of bound detection agents. None of the methods or compositions proposed to date has been demonstrated to be successful in complete removal of a bound agent, or in removal techniques that can allow for multiple rounds of staining.

Previously proposed formulations have been evaluated by the present inventors; however, none has been found to be successful. For example, a proposed composition of pH 2 glycine, SDS/2BME, and hot 6M urea (Gendusa et al., J Histochem Cytochem, 2014, 62(7):519-31) and a proposed composition of 15′ of ammonium thiocyanate pH 6 at 4 M (Pullen et al., J Immunol Methods, 1986, 86(1):83-7), done in ELISA, were found to result in incomplete removal of detection agents. In a proposed composition at high or low pH, 100 mM BME, 3.5 M KCl, and 1% SDS (Pirici et al., J Histochem and Cytochem, 2009, 57(6): 567-575), surfactants seemed to have little impact, as chaotropic salts seemed to remove the majority (but not all) of the staining. In a proposed composition of SCN > CLO4 > I-, at pH 4 (Dandliker et al., Biochemistry, 1967, 6(5):1460-7), chaotropic salts alone were found to be insufficient. On analysis of physical factors that allegedly contribute to antigen-antibody binding (Reverberi and Reverberi, Blood Transfus, 2007, 5(4):227-240), pH seemed to have little impact. The system of Ehrenberg et al. (J Neurosci Methods, 2020, 339:108708 and the “Denaturing Solution” provided by Biocare (Pacheco, CA) were similarly evaluated, along with numerous others, without success.

A specific example of sodium iodide and potassium iodide was tested as a denaturing agent, with sodium iodide at 1.84, 0.92, and 0.46 g/mL, and potassium iodide saturation points of 1, 0.5, and 0.25 g/mL, tested with 35 mg/mL tris(hydroxypropyl)phosphine (THPP) for 15 minutes as a reducing agent. In this comparative example, potassium iodide did not provide sufficient removal of antibody reagents, and sodium iodide was found not to be as effective as ammonium thiocyanate alone. Follow-up investigation found that sodium iodide quenched fluorescent dyes rather than stripping detection reagents.

None of the recipes and formulations reported in the literature is sufficient to strip off detection reagents as well as microwave treatment (MWT). However, MWT has the drawback of destroying or otherwise adversely impacting many types of tissue samples.

The present invention thus relates to compositions which are capable of stripping detection reagents, and methods involving such compositions. In particular embodiments, the compositions of the present invention can strip detection reagents without destroying or otherwise adversely impacting many types of tissue samples.

Indeed, particular embodiments of the disclosure relate to compositions which preserve tissue integrity and antigenicity through at least 6 rounds of antibody stripping and are capable of being formulated in a ready to use, room temperature or refrigerated reagent kit. The compositions remove antibodies and other non-covalently bound detection reagents.

The invention may be embodied in a variety of ways. In one aspect, the invention provides a composition for removing bound detection agents from a surface comprising: (i) a reductant and (ii) a denaturant. In particular embodiments, the composition can act as a stripping reagent for antibody removal / antibody stripping /antibody elution in immunofluorescence studies. Certain such compositions are also referred to herein as detection agent removal reagents (DARRs), antibody removal reagents (ARRs), VRK-1000, VectaPlex, etc.

In some embodiments, the surface is a stained biological sample.

In some embodiments, the biological sample is a tissue. In certain instances, the tissue comprises lung, liver, colon, heart, nervous system, skin, lymph gland, musculoskeletal, smooth muscle, spleen, eye, sinus, nasal mucosa, larynx, gastrointestinal tract tissue, reproductive organ, breast, prostate, salivary gland, tonsil, brain, connective tissue, epithelium, cardiovascular, or kidney.

In certain instances, the biological sample is immunostained. In other embodiments, the biological sample is histochemically stained. In some embodiments, the target nucleic acid in the sample can be hybridized with a probe (e.g., for use with in situ hybridization (ISH)). In some embodiments, the biological sample is a stained sample, such that the sample comprises bound detection agents. In certain embodiments, the bound detection agent is a dye, antibody, nanobody, lectin, recombinant protein, antibody fragment or fragments (e.g., Fab), streptavidin, avidin, dendrimer, enzyme (such as horse radish peroxidase, alkaline phosphatase, or glucose oxidase) or any combination thereof.

In some embodiments, the surface is a sensor or electrode. In some embodiments, the sensor or electrode is made of metal. In some embodiments, the metal sensor is silver, gold, platinum, palladium, titanium, and rhodium. In some embodiments, the metal is oxidized. In some embodiments, the sensor has a binding agent attached to it. In some embodiments, the binding agent is includes a protein, antibodies, nanobodies, lectins, glycan binding proteins, recombinant proteins, antibody fragments (e.g., Fab), streptavidin, avidin, dendrimer, peptide, enzyme such as horse radish peroxidase, alkaline phosphatase, or glucose oxidase, oligonucleotide, or any combination thereof. In this way, use of the detection agent removal reagent can be used to recycle or clean a sensor for subsequent or repeated use.

In some embodiments, the surface is functionalized with reactive groups. For example, the surface can be layered with hydrogels in order to bind one or more analytes, such as, for example, proteins, nucleic acids, and the like, via a chemistry process that uses one to many cycles of detection and removal. In this way, use of the detection agent removal reagent can be used to prime a surface for a sequential chemistry process.

A bound detection agent can be any reagent that is bound directly or indirectly to a specific target molecule. A bound detection agent is capable of providing a readout of the presence or absence of the target molecule, when coupled or used with another detection agent or when used alone. Non-limiting examples of bound detection agents can include a wide range of molecules such as antibodies, lectins, oligonucleotides, dyes, aptamers, proteins such as zinc fingers, or other molecules that bind specifically to targets as well as other non-limiting examples provided herein.

Antibodies can be immunoglobulin molecules or immunologically active portions (e.g., binding fragments) of immunoglobulin molecules (e.g., molecules that contain an antigen binding site that specifically binds an antigen). Antibodies, portions thereof (e.g., binding portions), or mutants or chimeras thereof can be expressed and/or isolated from any suitable biological organism or source. Non-limiting examples of antibodies include monoclonal antibodies, polyclonal antibodies, Fabs, Fab′, single chain antibodies, synthetic antibodies, dendrimers, peptides, polypeptides, or combinations thereof. A variety of antibodies and antibody fragments can be generated for use as a specific binding agent. Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized, or bispecific versions of such antibodies. In some embodiments a binding/capture agent used herein is an antibody, or fragment thereof that specifically binds an epitope or antigen. Polyclonal antibodies, monoclonal antibodies, fragments thereof, and variants thereof that bind specific antigens are commercially available, and methods for generating such antibodies are known.

In some embodiments, the bound detection agent is a probe. For example, the probe may be a probe used for ISH. ISH can be used to indicate the location of a nucleic acid sequence in a biological sample. Probes used for ISH may include but are not limited to oligonucleotide probes, single-stranded DNA probes, double-stranded DNA probes, branched DNA probes, RNA probes, and chimeric RNA/DNA probes. In some embodiments, the probes may be fluorescently labeled for fluorescence in situ hybridization (FISH) or chromogenically labeled for chromogenic in situ hybridization (CISH). In some embodiments, the probes may be labeled with an affinity reagent such as biotin that can be used to subsequently attach a detection reagent.

In some embodiments, the bound detection agent is a lectin or other glycan binding agent. Lectins are proteins that bind to carbohydrates and can be useful in the histochemical analysis of tissues to characterize the distribution of glycans.

In some embodiments, the bound detection agent is an enzyme (such as horse radish peroxidase, alkaline phosphatase, beta galactosidase, or glucose oxidase). An array of chromogenic, fluorogenic, and chemiluminescent substrates are available for use with such enzymes.

In some embodiments, the bound detection agent comprises a protein. The bound detection agent can be a protein and include one or more antibodies. In some embodiments, the antibody is a primary antibody. In some embodiments, the antibody is a secondary antibody. In some embodiments, the bound protein is a glycan binding reagent or protein. In certain instances, one or more of the bound proteins may be labelled. For example, in certain embodiments, the label is a dye, an enzyme, a mass tag, a nanoparticle, a quantum dot, or an oligonucleotide.

In some embodiments, the composition comprises a reducing agent (“reductant”). A reductant is any chemical that reduces another chemical by donating electrons. In some embodiments, the reductant reduces thiol bonds of the bound detection agents (e.g., proteins and dyes) by donating electrons. In certain instances, the reducing agent is a disulfide reducing agent. Reducing agents can be used to stabilize sulfhydryls (cysteines) and to reduce disulfide bonds in peptides and proteins. In some embodiments, the reductant is beta-mercaptoethanol, Lithium aluminum hydride (LiAlH4), Red-Al (NaAlH2(OCH2CH2OCH3)2), Nascent (atomic) hydrogen, Hydrogen without or with a suitable catalyst e.g. a Lindlar catalyst, Sodium amalgam (Na(Hg)), Sodium-lead alloy (Na + Pb), Zinc amalgam (Zn(Hg)), Diborane Sodium borohydride (NaBH4), Compounds containing the Fe2+ ion, such as iron(II) sulfate, Compounds containing the Sn2+ ion such as tin(II) chloride, Sulfur dioxide, Sulfite compounds, Dithionates (Na2S2O6), Thiosulfates (Na2S2O3), Iodides (KI), Hydrogen peroxide (H2O2), Hydrazine (Wolff-Kishner reduction), Diisobutylaluminium hydride (DIBAL-H), Oxalic acid (C2H2O4), Formic acid (HCOOH), Ascorbic acid (C6H8O6), Reducing sugars, Phosphites, hypophosphites, and phosphorous acid, Dithiothreitol (DTT), Carbon monoxide (CO), Cyanides in hydrochemical metallurgical processes, Carbon (C), tris-(hydroxypropyl)phosphene (THPP), and/or Tris-2-carboxyethylphosphine hydrochloride (TCEP), and the like.

In some embodiments, the composition of the reductant is between pH 2 and pH 10. In some embodiments, the composition may further comprise the reductant is between 1 uM and 1 M in concentration. In some embodiments, the reductant is present at a concentration of between 0.1 mg/mL and 100 mg/mL, or any sub-range within this range. In some embodiments, the reductant is present at a concentration of about 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 35 mg/mL, or higher. In some embodiments, the reductant is present at a concentration of about 5 mg/mL. In some embodiments, the reductant comprises THPP and is present in aqueous solution at a concentration of between 0.1 mg/mL and 35 mg/mL. In some embodiments, the reductant comprises THPP and is present in aqueous solution at a concentration of about 5 mg/mL.

In certain embodiments, the composition comprises a denaturant. A denaturant is any substance that causes denaturation of proteins, nucleic acids, or other biological compounds. For example, a denaturant is any substance that alters the original state of the protein or nucleic acid by chemical or physical means. In some embodiments a denaturing agent (“denaturant”) is chaotropic, or a detergent, or an acid, or a base, or a solvent, or a cross-linking reagent, or a disulfide bond reducer or any combination thereof. Denaturants can disrupt water interactions and promote hydrophobic protein and peptide solubilization, elution, refolding, and structural reorganization. In some embodiments, the denaturant is a chaotropic agent. A chaotropic agent is any substance which disrupts the three-dimensional structure of macromolecules including but not limited to proteins, DNA, or RNA. Chaotropic agents include urea, guanidium chloride, guanidinium thiocyanate (also referred to herein as guanidine thiocyanate) (GTC)/, ammonium thiocyanate, lithium perchlorate, sodium dodecyl sulfate, and the like. In some embodiments, the denaturant is an acid. Acids include acetic acid, trichloroacetic acid, and sulfosalicylic acid. In some embodiments, the denaturant is a base. Bases can include sodium hydroxide, sodium bicarbonate, potassium hydroxide, or lithium hydroxide to name a few. In some embodiments, the denaturant is a cross-linking reagent. Cross-linking reagents include formaldehyde and glutaraldehyde. In some embodiments, the denaturant is a disulfide bond reducer. Disulfide bond reducers include 2-mercaptoethanol, dithiothreitol, Tris-hydroxy propyl phosphene, and TCEP. Thus, in some embodiments, the denaturing agent includes ammonium thiocyanate, guanidinium thiocyanate / guanidine thiocyanate (GTC), urea, guanidine, guanidine chloride, lithium perchlorate, sodium perchlorate, sodium dodecyl sulfate, acetic acid, trichloroacetic acid, sulfosalicylic acid, sodium bicarbonate, dodecaborate (B₁₂H₁₂)⁻², ethanol, formaldehyde, glutaraldehyde, and/or the like.

In some embodiments, the composition of the denaturant is between pH 2 and pH 10. In some embodiments, the composition of the denaturant is between 1 mM and 10 M or at the saturation limit of the material, or any sub-range within this range. In some embodiments, the denaturant is present at a concentration of between 0.1 M and 6 M, or any sub-range within this range. In some embodiments, the denaturant is present at a concentration of about 0.1 M, 0.5 M, 1.0 M, 1.5 M, 2.0 M, 2.5 M, 3.0 M, 3.5 M, 4.0 M, 4.5 M, 5.0 M, 5.5 M, 6.0 M, or greater. In some embodiments, the denaturant is present at a concentration of about 295 mg/mL. In some embodiments, the denaturant comprises GTC and is present in aqueous solution at a concentration of 2.5 M. In some embodiments, the denaturant comprises GTC and is present in aqueous solution at a concentration of about 295 mg/mL.

In some embodiments, the composition comprises a reductant and a denaturant. In some embodiments, the reductant comprises THPP and is present in aqueous solution at a concentration of between about 1 to 100 mg/mL, or any sub-range within this range, and the denaturant comprises GTC and is present in aqueous solution at a concentration of between about 1 M to 6 M, or any sub-range within this range.

### Multiplex Sequential Staining

Multiplex immunofluorescence workflows allow the detection of multiple markers within a single specimen. Commonly, this is achieved using primary antibodies of different species or using primary antibodies that are directly labelled as to avoid the cross reactivity that can occur between immunofluorescence detection reagents. Multiplexing provides specific advantages over single-plex assays: 1) less precious sample is utilized (instead of one tissue used per marker, one tissue is used for all markers), 2) exponential increase in experimental data as one can now see the expression, spatial relationship and distribution of multiple markers within a single specimen, Limitations in multiplexing come from two predominant sources, 1) spectral overlap of dyes limits the number of colors that can be visualized, and 2) each primary antibody needs to be from a different species so that secondary antibodies can be utilized without cross-reactivity. These aspects have limited the plexy levels of tissue staining to be quite low and are usually not more than 3-plexy due to the species limitation. Most primary antibodies are made in mouse or rabbit and thus, use of multiple mouse or rabbit primaries with the corresponding secondary antibodies can lead to non-specific cross-talk between the signals generated from the different primary antibodies.

DARR overcomes these limitations by allowing removal or stripping of each round of staining to allow sequential staining and subsequent merging of the signal generated from each marker into a single image. This prevents any crosstalk between two or more primaries that are from the same species. Also, since each round of staining is performed individually, the same color or detection modality can be used and thus preventing any issues that can arise from spectral overlap.

Another aspect of the present invention provides a method for removal of bound detection agents from stained samples. In some embodiments, the method for removing bound detection agents from a stained sample allows for a multiplex sequential staining of a sample. In some cases, the methods can use heat, microwaves, and/or pressure cookers to facilitate the release of bound detection agents.

### Samples

In some embodiments, the sample for the methods described herein is a biological sample. In certain instances, the biological sample is a tissue. In some embodiments, the tissue comprises lung, liver, heart, nervous system, skin, lymph gland, musculoskeletal, smooth muscle, colon, spleen, eye, sinus, nasal mucosa, larynx, gastrointestinal tract tissue, reproductive organ, breast, prostate, salivary gland, tonsil, brain, connective tissue, epithelium, cardiovascular, or kidney tissue.

In certain embodiments, the sample comprises a marker or analyte of interest. In some embodiments, the marker is intracellular. In other embodiments, the marker is extracellular. In some embodiments, the marker is a membrane protein. In some embodiments, the marker is a cytoplasmic protein. In some embodiments the marker is an oligonucleotide. In certain embodiments, the marker is a carbohydrate. In certain embodiments, the marker is a nuclear protein.

In some embodiments, the sample has been preserved. For example, tissue samples may be preserved to prevent the breakdown of cellular protein and degradation of the normal tissue architecture. In some embodiments, the sample is a preserved tissue sample. In some instances, the sample is a fresh or unfixed sample. In other instances, the sample has been fixed. Any method of fixation known in the art may be used with the embodiments herein. The fixation method may be selected based on the sample type, target antigen, and application. For example, in some embodiments, the sample is formalin-fixed and paraffin embedded (FFPE). Formalin-fixed tissue samples are usually embedded in paraffin to maintain their natural shape and tissue architecture during long-term storage and to facilitate sectioning. Thus, in some embodiments, the sample is FFPE. In some embodiments, the sample is deparaffinized prior to staining and subsequent removal of bound detection agents.

In some embodiments samples are too sensitive to degradation for chemical fixation. In certain embodiments, the sample is an unfixed sample. In certain instances, the sample is an unfixed frozen sample or cryosection. For example, a tissue can be embedded in a cryogenic substance and then frozen in liquid nitrogen. Frozen samples can be sectioned using a cryostat, transferred to slides, and then dried to preserve morphology. In some instances, the sample is a fixed, frozen sample. For example, a tissue sample may be fixed then cryoprotected with a stabilizer prior to freezing and sectioning; for example, a tissue sample may be sectioned and then fixed in acetone.

### Stained Samples

In some embodiments, the sample for the methods described herein is a stained sample, such that the sample contains bound detection agents. In certain embodiments, the bound detection agent is a dye or enzyme. In certain embodiments, the bound detection agent is an antibody, nanobody, lectin, recombinant or natural protein, antibody fragment (e.g., Fab), streptavidin, avidin, dendrimer, oligonucleotide, peptide, or any combination thereof.

In some embodiments, the bound detection agent is one or more antibody. In certain embodiments, the antibody is a primary antibody. In some embodiments, the bound detection agent is a secondary antibody. Antibodies can be immunoglobulin molecules or immunologically active portions (e.g., binding fragments) of immunoglobulin molecules (e.g., molecules that contain an antigen binding site that specifically binds an antigen). Antibodies, portions thereof (e.g., binding portions), mutants or chimeras thereof can be expressed and/or isolated from any suitable biological organism or source. Non-limiting examples of antibodies include monoclonal antibodies, polyclonal antibodies, Fabs, Fab′, single chain antibodies, synthetic antibodies, dendrimers, peptides, polypeptides, or combinations thereof. A variety of antibodies and antibody fragments can be generated. Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of such antibodies. Antibodies, monoclonal antibodies, polyclonal antibodies, fragments thereof, and variants thereof that bind specific antigens are commercially available, and methods for generating such antibodies are known.

Fluorescent labeling relies on conjugation of the detection agent with a fluorophore or other molecule. These fluorescent molecules emit light within specific ranges between 300 and 900 nm when excited by a light source. Given the full range of emission wavelengths within the useable spectra, multiple fluorescent markers can be used simultaneously for detailed co-localization characterizations. In some embodiments, the fluorophore can be conjugated to the detection agent. For direct detection when the detection agent is an antibody, the fluorophore is conjugated to the primary antibody. For indirect detection when the detection agent is an antibody, the fluorophore is conjugated to the secondary antibody.

In some embodiments, the stained sample contains a bound detection agent, wherein the bound detection agent is a probe to the target marker of interest (e.g. proteins, carbohydrates or nucleic acids). In some embodiments, the stained sample comprises a sample labelled with a fluorescent probe. In certain embodiments, the stained sample comprises a sample labelled with a chromogenic label. In certain embodiments, the stained sample contains a label produced by an enzyme. For example, the sample may be stained using tyramide signal amplification (TSA). TSA labeling involves binding of a probe to the target marker (e.g., proteins, carbohydrates or nucleic acids) followed by secondary detection of the probe with an HRP-labeled antibody or streptavidin conjugate. With TSA staining, the horseradish peroxidase (HRP) enzyme covalently attaches the dyes to protein moieties in the vicinity of the target molecule. In this modality, the fluorescent label remains when using reagents described herein that remove the detection agent. Thus, through subsequent rounds, multiple different colors can be attached to the sample, each using a different target molecule to bind the detection agent. In some embodiments, probes may be used in either direct or indirect format.

Chromogenic labeling relies on chemical reactions triggered by enzymes conjugated to a detection agent such as with either a primary or secondary antibody. Peroxidases such as HRP are a commonly used tool for such reactions. Once added, liquid substrate reacts with these enzymes and forms a solid that precipitates out of solution and produces a color deposited at the target site. For enzyme labels used in TSA methodologies, the enzyme can alter the form of the dye so that it covalently attaches itself (the dye) to nearby tyrosine amino acids in the sample. Enzymes include but are not limited to horseradish peroxidase (HRP), alkaline phosphatase (AP), beta galactosidase, and glucose oxidase. For example, HRP can convert substrates including 3,3′-diaminobenzidine (DAB) into a brown product and AP can convert substrates including 3-amino-9-ethylcarbazole (AEC) into a red product. In some embodiments, the sample comprises one or more different colored chromogens.

In certain instances, the bound detection agent is labelled. For example, in certain embodiments, the label is a dye, an enzyme, a mass tag, a nanoparticle, a quantum dot, or an oligonucleotide. In some embodiments, the detection label is a fluorescent molecule (e.g., a fluorescent dye), a fluorogenic molecule, an oligonucleotide, or a metal. Optionally, the mode of detection is through a fluorogenic label. A fluorogenic label can be any label that is capable of emitting light when in an unquenched form (e.g., when not quenched by another agent). The fluorescent moiety emits light energy (i.e., fluoresces) at a specific emission wavelength when excited by an appropriate excitation wavelength. When the fluorescent moiety and a quencher moiety are in close proximity, light energy emitted by the fluorescent moiety is absorbed by the quencher moiety. In some embodiments, the fluorogenic dye is a fluorescein, a rhodamine, a phenoxazine, an acridine, a coumarin, or a derivative thereof. In some embodiments, the fluorogenic dye is a carboxyfluorescein. Further examples of suitable fluorogenic dyes include the fluorogenic dyes commercially available under the ALEXA FLUOR™ product line (Life Technologies; Carlsbad, CA) and DyLight™ product line (Thermo Fisher Scientific; Waltham, MA). Optionally, the label is a reduction tag, a thio-containing molecule, or a substituted or unsubstituted alkyl. Optionally, the label is a fluorescent protein such as phycoerythrin, green fluorescent protein, or yellow fluorescent protein.

In some embodiments, the stained sample comprises a plurality of bound detection agents specific to 1, 2, 3, 4, 5, 6, 7, 8, 9, 10 or more unique target markers. In some embodiments, the stained sample comprises a plurality of bound detection agents specific to between 10 and 100 or more unique target markers. In some embodiments, each bound detection agent specific to a unique target marker comprises a unique label. In some embodiments, each bound detection agent specific to a unique target marker comprises a similar label used in previous rounds of sequential multiplexing.

### Removal of Bound Detection Agents

The method described herein further comprises removing one or more bound detection agents from a stained sample or surface.

In some embodiments, the removing step comprises applying a reductant to the stained sample or surface. Reductants that can be used in the methods described herein are described above.

In some embodiments, the reductant is added for less than 100, 90, 80, 70, 60, 50, 40, 30, 20, 25, 20, 15, 10, 9, 8, 7, 6, 5, 4, 3, 2, or 1 minutes.

In some embodiments, the removing step comprises applying a denaturant to the stained sample or surface. Denaturants that can be used in the methods described herein are described above. In some instances, the denaturant is chaotropic. A denaturant can disrupt water interactions and promote hydrophobic protein and peptide solubilization, elution, refolding, and structural alterations. A chaotropic agent is any substance which disrupts the three-dimensional structure in macromolecules including but not limited to proteins, DNA, or RNA. In some embodiments, the denaturant is ammonium thiocyanate, guanidinium/guanidine thiocyanate, urea, guanidine, guanidine chloride, lithium perchlorate, sodium perchlorate, sodium dodecyl sulfate, acetic acid, trichloroacetic acid, sulfosalicylic acid, sodium bicarbonate, dodecaborate (B₁₂H₁₂)⁻², ethanol, formaldehyde, or glutaraldehyde.

In some embodiments, the denaturant is added for less than 100, 90, 80, 70, 60, 50, 40, 30, 20, 25, 20, 15, 10, 9, 8, 7, 6, 5, 4, 3, 2, or 1 minutes. In some embodiments, the denaturant is added multiple times in a row to the same sample or surface.

In certain embodiments, the stained sample or surface is incubated with the reductant and/or denaturant at a first temperature. In some cases, the first temperature can be room temperature. In some cases, the first temperature can be between 25° C. and 45° C. In some cases, the first temperature can be greater than room temperature. In some cases, the first temperature can be between 70° C. to 130° C., such as 75° C., 80° C., 85° C., 90° C., 95° C., 100° C., 105° C., 110° C., 115° C., 120° C., 125° C., and/or 130° C., or any subranges thereof, such as 75° C. to 125° C., 80° C. to 120° C., 85° C. to 115° C., 90° C. to 110° C., and/or 95° C. to 105° C. In some cases, a microwave may be used to incubate the stained sample or surface to the first temperature. In some cases, a pressure cooker may be used to incubate the stained samples to the first temperature.

In some instances, the denaturant is applied to the stained sample or surface simultaneously with the reductant. In certain instances, the denaturant reagent is mixed with the reductant reagent to form a detection agent removal reagent. In some embodiments, the detection agent removal reagent is applied to the stained sample or surface. Thus, in certain embodiments, a composition comprising a denaturant and a reductant is added to the stained sample or surface. In other embodiments, the denaturant is added to the stained sample or surface following incubation with a reductant. In certain embodiments the stained sample or surface is washed with the denaturant. In some embodiments, the stained sample or surface is removed from the reductant prior to application of the denaturant. In certain embodiments the stained sample or surface is washed with the reductant. In other embodiments, the denaturant is applied to the stained sample or surface before the reductant is applied. In some embodiments, the stained sample or surface is removed from the denaturant prior to the application of the reductant. Optionally, following incubation with the denaturant and/or reductant, excess reagents are removed (e.g., washed away) from the sample. In some embodiments, the wash reagent is PBS.

In some embodiments the method comprises removing bound detection agents from the stained sample or surface at least 1, 2, 3, 4, 5, 6, 7, 8, 9, or 10 times. In some embodiments, the method comprises removing bound detection agents from the stained sample between 10 and 100 times.

In some embodiments, the method removes over 50% of the total quantity of the one or more bound detection agent from the stained sample or surface. In some embodiments, the method removes over 60%, 70%, 75%, 80%, 85%, 90%, 91%, 92%, 93%, 94%, 95%, 96%, 97%, 98%, 99%, or more of the total quantity of the one or more bound detection agent from the stained sample or surface. In some embodiments, the method removes substantially all of the total quantity of the one or more bound detection agents from the stained sample or surface. In some embodiments, no bound detection agent can be detected from the stained sample or surface after treatment according to the methods as described herein.

### Staining

In some embodiments, the method further comprises an immunostaining step. Immunostaining refers to the use of proteins to detect a single marker or antigen of interest. In some embodiments, the immunostaining step is immunohistochemistry (IHC). IHC is performed on samples from tissues. IHC combines anatomical, immunological, and biochemical techniques to image discrete components in tissues by using appropriate antibodies, either labeled or unlabeled, to bind specifically to their target antigens in situ. IHC allows for the visualization of the distribution and localization of specific cellular components within cells and within their proper histological context.

In some embodiments, the method further comprises an immunostaining step. Immunostaining refers to the use of proteins to detect a single antigen of interest. In some embodiments, the immunostaining step is immunofluorescence (IF). IF is performed on samples from tissues. IF combines anatomical, immunological, and biochemical techniques to image discrete components in tissues by using appropriate antibodies, either labeled or unlabeled, to bind specifically to their target antigens in situ. IF allows for the visualization of the distribution and localization of specific cellular components within cells and within their proper histological context.

In some embodiments, the immunostaining step is immunocytochemistry (ICC). ICC is performed on samples consisting of cells grown in a monolayer or cells in suspension which are deposited on a slide, or other surface, such as a coverslip. Sample sources for ICC can be from any suspension of cells, obtained from patients or animals (e.g., blood smears, swabs, and aspirates) or cultured cells grown in monolayers.

In some embodiments, the staining can be ISH. ISH can be used to indicate the location of a nucleic acid sequence in a biological sample. In some embodiments, the ISH technique is FISH. In other embodiments, the ISH technique is CISH. FISH techniques rely on fluorescence, while CISH techniques combine chromogenic signal detection methods of immunohistochemistry (IHC) techniques with ISH. FISH allows for direct detection, whereas CISH methods use indirect detection methods. For example, CISH generally uses at least two additional steps beyond probe hybridization. In some embodiments for FISH detection, a probe can be labeled with FITC or rhodamine, and thereby detected directly using the FISH method. In other embodiments for CISH detection, the same probe might be labeled with biotin and then detected by sequential incubations with streptavidin-HRP and DAB, using the CISH method.

In certain instances, the sample is stained with one or more antibodies, nanobodies, lectins, glycan binding proteins, recombinant proteins, antibody fragments (e.g., Fab), streptavidin, avidin, dendrimer, peptide, or any combination thereof such that the detection agent binds to antigens of interest within the sample.

Although detection agents show preferential avidity and affinity for specific epitopes, detection agents may partially or weakly bind nonspecifically to sites on the sample that mimic the correct binding sites of the target molecule. In the context of antibody-mediated antigen detection, nonspecific binding causes high background staining that can mask the detection of the target antigen. To reduce background staining in IHC, IF, ICC, and any other staining application, prior to staining, the samples may be incubated with a buffer that blocks the non-specific sites to which the primary or secondary antibodies, or other detection agents, may otherwise bind. Common blocking buffers include some percentage of normal serum, non-fat dry milk, BSA (bovine serum albumin), gelatin, Carbo-Free block, Animal-free block, WestVision Block, and one or more gentle surfactants to aid in wetting. Many commercial blocking buffers with proprietary formulations are available for greater blocking efficiency.

In certain instances, the sample is stained with a labeled detection agent. For example, in certain embodiments, the label is a dye, an enzyme, a mass tag, a nanoparticle, a quantum dot, or an oligonucleotide. In some embodiments, the labeled detection agent is a fluorescent molecule (e.g., a fluorescent dye), a fluorogenic molecule, or a metal. Optionally, the detection agent is a fluorogenic label. The fluorescent moiety emits light energy (i.e., fluoresces) at a specific emission wavelength when excited by an appropriate excitation wavelength. When the fluorescent moiety and a quencher moiety are in close proximity, light energy emitted by the fluorescent moiety is absorbed by the quencher moiety. Optionally, the detection label is a fluorogenic dye. In some embodiments, the fluorogenic dye is a fluorescein, a rhodamine, a phenoxazine, an acridine, a coumarin, or a derivative thereof. In some embodiments, the fluorogenic dye is a carboxyfluorescein. Further examples of suitable fluorogenic dyes include the fluorogenic dyes commercially available under the ALEXA FLUOR™ product line (Life Technologies; Carlsbad, CA) and DyLight™ product line (Thermo Fisher Scientific; Waltham, MA). Optionally, the label is a reduction tag, a thiol-containing molecule, or a substituted or unsubstituted alkyl.

In certain instances, the sample is counterstained. Counterstains provide contrast to the primary stain and can be cell structure specific. These single-step stains are usually added after antibody staining. Common counterstains include hematoxylin, eosin, nuclear fast red, methyl green, 4′,6-diamidino-2-phenylindole (DAPI), and Hoechst fluorescent stain.

In some embodiments, the method comprises sequential staining with detection agents and bound detection agent removal steps. In some embodiments, the method comprises removing bound detection agents from the stained sample at least 1, 2, 3, 4, 5, 6, 7, 8, 9, or 10 times. In some embodiments the method comprises removing bound detection agents from the stained sample between 10 and 100 times.

### Detection/Imaging

In some embodiments, the method further comprises a detection step. In certain instances, the bound detection agent is labelled. For example, in certain embodiments, the label is a dye, an enzyme, a mass tag, a nanoparticle, a quantum dot, or an oligonucleotide.

Thus, in certain embodiments, the labeled bound detection agent is detectable. For example, detection of a primary antibody can be accomplished in multiple ways. The primary antibody can be directly labeled using an enzyme,fluorophore, tag, or mass-tag. The primary antibody can be labeled using a small molecule which interacts with a high affinity binding partner that can be linked to an enzyme or fluorophore. Biotin-streptavidin is one commonly used high affinity interaction. The primary antibody can be probed for using a broader species-specific secondary antibody that is labeled using an enzyme or fluorophore. In the case of electron microscopy, antibodies are linked to a heavy metal particle or mass-tag (typically gold nanoparticles in the range 5-15 nm diameter). Enzymes such as horseradish peroxidase or alkaline phosphatase are commonly used to catalyze reactions that give a colored or fluorescent product. Fluorescent molecules can be visualized using fluorescence microscopy or confocal microscopy. Fluorescence emissions from the label can be detected using techniques known to those skilled in the art, for example, the use of a light source emitting a specific wavelength in combination with filters. Exemplary detection methods include fluorescence microscopy, total internal reflection fluorescence microscopy, high inclined illumination microscopy, bright field microscopy, oblique illumination, dark field microscopy, parallel confocal microscopy, or mass spectrometry.

Detection agent-mediated antigen detection approaches are separated into direct and indirect methods. Both of these methods use detection agents to detect the target antigen, but the selection of the best method to use depends on the level of target antigen expression, its accessibility, the type of readout desired and the species of primary antibody available when using antibodies. Most indirect methods employ the inherent binding affinity of species-specific antibodies or strept(avidin) and related proteins for biotin to detect a bound antibody of known speciation or a biotinylated antibody that is bound to the target antigen. The antigen-bound antibody is then localized by adding an enzyme-conjugated secondary antibody or fluorophore conjugated secondary antibody which generates a signal when excited with the correct wavelength of light or an amplified signal when appropriate substrates are added.

Detecting the target molecule with detection agents can be a multi-step process that uses optimization at every level to maximize the signal. Optimization may include determining the appropriate diluent, concentration, and incubation time. Typically, detection agents are diluted into a buffer formulated to help stabilize the reagents, promote its uniform and complete diffusion into the sample, and discourage nonspecific binding. While one diluent may work with one detection agent, the same diluent may not work with another detection agent, demonstrating the need for optimization for each one. The concentration of the detection agents can also be optimized to balance specific and non-specific binding. The length of time the sample is exposed to the detection agent can also be optimized to balance specific and non-specific binding.

Rinsing the sample in between detection agent applications can remove unbound detection agents and remove detection agents that are weakly bound to nonspecific sites. In some cases, rinse buffers comprising selected and/or certain components can maximize washing efficiency and minimize interference with the signal.

The invention will be illustrated through the following series of specific embodiments. However, it will be understood by one skilled in the art that many other embodiments are contemplated by the principles of the invention.

### Sequential Multiplexing

In some embodiments, a sequential method for multiplex detection comprises: (i) identifying a plurality of target markers of interest in a biological sample; (ii) staining the biological sample with a first plurality of detection agents specific for a first plurality of target markers of interest; (iii) optionally, detecting a visual signal that indicates the presence or absence of the markers of interest from step (ii); (iv) removing the first plurality of bound detection agents from the stained biological sample; (v) staining the biological sample with a second plurality of detection agents specific for a second plurality of target markers of interest; (vi) optionally, detecting a visual signal that indicates the presence or absence of the markers of interest from step (v), and (vii) optionally, repeating steps (iv)-(vi) for detection of additional markers of interest.

In some embodiments, the method comprises identifying a plurality of target markers of interest in a biological sample. In some embodiments, a plurality of markers of interest is first determined. Markers of interest may include various components of a tissue or cell. In some embodiments, the marker is intracellular. In other embodiments, the marker is extracellular. In some embodiments, the marker is a membrane protein. In some embodiments, the marker is a cytoplasmic protein. In some embodiments the marker is an oligonucleotide or oligonucleotide sequence. In certain embodiments, the marker is a carbohydrate. In certain embodiments, the marker is a nuclear protein.

In certain embodiments, the method comprises staining the biological sample with a first plurality of detection agents. In some embodiments, one or more detection agents are incubated with the sample for a length of time sufficient to allow the detection agent(s) to bind to the correct antigen(s) in the sample. In some embodiments, the bound detection agent comprises at least one of dyes, antibodies, nanobodies, lectins, recombinant proteins, antibody fragments such as Fab, streptavidin, avidin, oligonucleotides, enzymes, or any combination thereof as described in detail herein. In some embodiments, the plurality of target markers comprises at least 1, 2, 3, 4, 5, 6, 7, 8, 9, 10, 15, 20, 25, 20, 35, 40, 45, or 50 markers of interest.

In some instances, the method comprises detecting a visual signal that indicates the presence or absence of the marker of interest. Any methods known in the art for detecting specific targets through binding interactions may be used with methods described herein. Exemplary detection methods include fluorescence microscopy, total internal reflection fluorescence microscopy, high inclined illumination microscopy, bright field microscopy, oblique illumination, dark field microscopy, or parallel confocal microscopy. In some embodiments, an image of the visual signal is captured.

In some embodiments, the method comprises removing a bound detection agent from the stained biological sample. In some embodiments, the method for removing bound detection agent comprises (i) applying a reductant to the stained sample; and (ii) applying a denaturant to the stained sample as described in detail herein. In some embodiments, the removal of the detection agent (e.g., antibody, dye) is confirmed by performing the detection steps. In certain instances, the absence of a signal confirms the removal of the detection reagent. In some embodiments, the visual signal remains following removal of the bound detection agent.

In certain embodiments, the method comprises staining the biological sample with a second plurality of detection agents. In some embodiments, one or more detection agents are incubated with the sample(s) for a length of time sufficient to allow the detection agent(s) to bind to the sample. In some embodiments, the bound detection agent(s) comprises at least one of dyes, antibodies, nanobodies, lectins, recombinant proteins, antibody fragments such as Fab, streptavidin, avidin, oligonucleotides, or any combination thereof as described in detail herein.

In some embodiments, following staining of the biological sample, the method optionally comprises detecting a visual signal that indicates the presence or absence of the marker of interest. Any methods known in the art for detecting the detection agent may be used with this invention. Exemplary detection methods include fluorescence microscopy, total internal reflection fluorescence microscopy, high inclined illumination microscopy, bright field microscopy, oblique illumination, dark field microscopy, or parallel confocal microscopy. In some embodiments, an image of the visual signal is captured. In certain embodiments, the image captured from incubation with a first plurality of detection agents is combined with the image captured from incubation with a second plurality of detection agents. In some cases, multiple images of the visual signal are captured after each round of detection agent application and the final images are combined.

In some embodiments, the method steps of removing the first plurality of bound detection agents from the stained biological sample; staining the biological sample with a second plurality of detection agents specific for a second plurality of target markers of interest; and optionally, detecting a visual signal that indicates the presence or absence of the markers of interest from staining the biological sample with a second plurality of detection agents are repeated at least 1, 2, 3, 4, 5, 6, 7, 8, 9, or 10 or more times. Thus, the methods described herein allows for sequential multiplex detection.

In some embodiments, the imaging step is performed sequentially. In some embodiments, the method utilizes the detection and capture of the visual signal after each round of staining. In other embodiments, the visual signal remains on the sample following removal of the detection agent. In certain embodiments, the methods only utilize detection and capture of the visual signal following staining the biological sample with the last plurality of detection agents. Thus, in some embodiments, the imaging step is performed once at the end of labelling with multiple fluorophores or chromogens. In sequential imaging, the stained sample is imaged after each labelling step of the first set of plurality of markers, where imaging is usually done after staining in each round and before removal of detection agents. Imaging can also be done between the use of the detection agent removal reagent and the addition of subsequent detection agents. Depending on how many markers are to be identified, the final set of images are then combined or correlated into a single image or analysis. When imaging is performed at the end, such as what would be done when using tyramide signal amplification (TSA), all the labels from the plurality of markers to be detected are present on the tissue for the imaging step at the end. For example, the detection agent removal reagent does not remove the covalently attached dyes of TSA, only the enzymes used to attach the dyes to the stained sample are removed. In this embodiment, while the removal process is performed between each marker, there is no image taken until all the dyes have been attached to the sample.

In some embodiments, the method does not involve capturing an image after each round of staining (such as, for example, when using TSA techniques). Thus, in certain embodiments provided herein is a method for multiplex detection comprising: (i) identifying a plurality of target markers of interest in a biological sample; (ii) staining the biological sample with a first plurality of detection agents specific for a first plurality of target markers of interest; (iii) removing the first plurality of bound detection agents from the stained biological sample; (iv) staining the biological sample with a second plurality of detection agents specific for a second plurality of target markers of interest; (v) optionally, repeating steps (iii)-(iv) for detection of additional markers of interest; and (vi) detecting a visual signal that indicates the presence or absence of the markers of interest.

### Systems and Kits

In certain embodiments, the disclosure provides systems for performing the methods disclosed herein and/or using the compositions described herein. In certain embodiments, the system may comprise a kit. In some cases, the system may comprise automated instructions and/or reagents for performing the methods disclosed herein.

For example, in some embodiments, the system may comprise one or more of the following: a station for providing a sample (e.g., a biological sample) believed to contain at least one marker of interest; optionally, a station for staining at least one marker of interest in the sample using a detection agent; a station for detecting the marker of interest; a station to analyze the results to determine the presence or amount of the one or more markers in the sample; a station for removing the detection agents; and processes by which to repeat the various steps in sequence. Also in certain embodiments, at least one of the stations is automated and/or controlled by a computer.

In certain embodiments, the disclosure provides kits for use in accordance with methods and compositions disclosed herein. In some embodiments, kits comprise one or more agents for the removal of detection agents as described herein. In certain instances, the kits further comprise one or more detection agents. In some embodiments the reagents for the removal of detection agents comprises a denaturant and/or a reductant.

### EXAMPLES

The following non-limiting examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

### Example 1

### Exemplary Imaging Methods and Protocols

### Protocol 1: Tissue Deparaffinization and Rehydration

1) FFPE tissue sections are deparaffinized and rehydrated by incubating them in sequential washes of:
a) Xylene for 10 minutes
b) Xylene for 10 minutes
c) 100% ethanol for 3 minutes
d) 100% ethanol for 3 minutes
e) 100% ethanol for 3 minutes
f) 95% ethanol for 3 minutes
g) 95% ethanol for 3 minutes
h) 70% ethanol for 3 minutes
i) Tap water for 10 minutes
j) 1× PBS for 3 minutes

### Protocol 2: Tissue Dehydration and Clearing

FFPE tissue sections are dehydrated and cleared by placing them in the following, sequentially:
a) Tap water for 3 minutes
b) 95% ethanol for 2 minutes
c) 95% ethanol for 2 minutes
d) 100% ethanol for 2 minutes
e) 100% ethanol for 2 minutes
f) Clear-Rite (Kalamazoo, MI) for 3 minutes
g) Clear-Rite for 3 minutes
h) Clear-Rite for 3 minutes
i) Mounting media and coverslip are then applied.

### Protocol 3: Tissue Staining - Immunofluorescence Workflow

The tissue staining immunofluorescence workflow is as in the following sequence:

1) Rehydrated tissue is placed into a pressure cooker that is filled with 1× Antigen Unmasking Solution, Citrate-Based (Vector Labs, Newark), and allowed to pressure cook for 1 minute at maximum pressure.

2) Non-specific binding in tissue sections is blocked by incubating with 2.5% Normal Horse Serum (2.5% NHS) for 30 minutes.

3) The block is tipped-off.

4) Primary binders, including primary antibodies, are applied to tissue sections. Primary binders are usually diluted in dilute animal serum to minimize off-target binding (i.e. 2.5% NHS). Each primary binder has a unique dilution amount for optimal staining.

5) Tissue sections are washed for 5 minutes with 1× PBS.

6) Dye-labelled secondary antibodies, which are directed against the species of the primary antibody, are applied at 10 ug/mL diluted in 1 × PBS or 2.5% NHS and incubated for 30 minutes.

7) Tissue sections are washed for 5 minutes with 1× PBS.

8) TrueView Autofluorescence Quenching Reagent (Vector Labs, Newark) is applied to the tissue sections for 5 minutes to minimize the autofluorescence.

9) Tissue sections are washed for 5 minutes with 1× PBS.

10) Tissue sections are mounted with 25 uL of VECTASHIELD Vibrance® Antifade Mounting Medium with DAPI (Vector Labs, Newark) and a coverslip to prevent fading and preserve the fluorescent signal.

11) Tissue sections are allowed to cure at room temperature for 30 minutes before imaging.

12) Imaging is performed on a Nikon fluorescent microscope.

### Protocol 4: Tissue Staining - TSA Workflow

The tissue staining TSA workflow is as in the following sequence:

1) Rehydrated tissue is placed into a pressure cooker that is filled with 1× Antigen Unmasking Solution, Citrate-Based (Vector Labs, Newark) and allowed to pressure cook for 1 minute at maximum pressure.

2) Endogenous peroxidase activity is blocked in tissue sections by applying Bloxall Endogenous Blocking Solution (Vector Labs, Newark) for 10 minutes.

3) Tissue sections are washed for 5 minutes with 1× PBS.

4) Non-specific binding in tissue sections is blocked by incubating with 2.5% Normal Horse Serum (2.5% NHS) (Vector Labs, Newark) for 30 minutes.

5) The block is tipped-off.

6) Primary binders, including primary antibodies, are applied to tissue sections. Primary binders are usually diluted in dilute animal serum to minimize off-target binding (i.e. 2.5% NHS). Each primary binder has a unique dilution amount for optimal staining.

7) Tissue sections are washed 3 × 2 minutes each with 1× TBST.

8) Tissue sections are incubated with HRP-labelled secondary antibodies in 1× PBS for 10-30 minutes (10 minutes using Akoya secondaries and 30 minutes for Vector secondaries).

9) Tissue sections are washed 3 × 2 minutes each with 1x TBST.

10) TSA substrates are diluted according to the vendor’s instructions and applied to tissue sections for 10 minutes.

11) Tissue sections are washed 3 × 2 minutes each with 1× TBST.

12) Tissue sections are mounted with 25 uL of VECTASHIELD Vibrance® Antifade Mounting Medium with DAPI (Vector Labs, Newark) and a coverslip.

13) Tissue sections are allowed to cure at room temperature for 30 minutes before imaging.

14) Imaging is performed on a Nikon fluorescent microscope.

### Protocol 5: Tissue Staining - Immunofluorescence Workflow With DARR (Detection Agent Removal Reagent) Stripping Step(s).

The tissue staining immunofluorescence workflow with one DARR stripping step is as in the following sequence:

1) Rehydrated tissue is placed into a pressure cooker that is filled with 1× Antigen Unmasking Solution, Citrate-Based (Vector Labs, Newark) and allowed to pressure cook for 1 minute at maximum pressure.

2) Non-specific binding in tissue sections is blocked by incubating with 2.5% Normal Horse Serum (2.5% NHS) for 30 minutes.

3) The block is tipped-off.

4) Primary binders, including primary antibodies, are applied to tissue sections. Primary binders are usually diluted in dilute animal serum to minimize off-target binding (i.e. 2.5% NHS). Each primary binder has a unique dilution amount for optimal staining.

5) Tissue sections are washed for 5 minutes with 1× PBS.

6) Dye-labelled secondary antibodies, which are directed against the species of the primary antibody, are applied at 10 ug/mL diluted in 1× PBS or 2.5% NHS and incubate for 30 minutes.

7) Tissue sections are washed for 5 minutes with 1× PBS.

8) TrueView Autofluorescence Quenching Reagent (Vector Labs, Newark) is applied to the tissue sections for 5 minutes to minimize the autofluorescence.

9) Tissue sections are washed for 5 minutes with 1× PBS.

10) Tissue sections are mounted with 25 uL of VECTASHIELD Vibrance® Antifade Mounting Medium with DAPI (Vector Labs, Newark) and a coverslip to prevent fading and preserve the fluorescent signal.

11) Tissue sections are allowed to cure at room temperature for 30 minutes before imaging.

12) Imaging is performed on a Nikon fluorescent microscope.

13) Slides are unmounted by soaking and rinsing them in 1× PBS to help remove the coverslip.

14) Detection agents are stripped off the tissue sections using DARR Reagent 1 for 15 minutes, a rinse of 1x PBS followed by 15 minutes incubation of DARR Reagent 2.

15) Tissue sections are washed for 5 minutes with 1× PBS.

16) If no more rounds of staining are desired, proceed to the next step. For additional staining rounds, repeat the above steps starting from step 2.

17) TrueView Autofluorescence Quenching Reagent (Vector Labs, Newark) is applied to the tissue sections for 5 minutes to minimize the autofluorescence.

18) Tissue sections are washed for 5 minutes with 1× PBS.

19) Tissue sections are mounted with 25 uL of VECTASHIELD Vibrance® Antifade Mounting Medium with DAPI (Vector Labs, Newark) and a coverslip to prevent fading and preserve the fluorescent signal.

20) Tissue sections are allowed to cure at room temperature for 30 minutes before imaging.

21) Imaging is performed on a Nikon fluorescent microscope.

### Protocol 6: Tissue Staining - TSA With DARR Stripping Step(s).

The tissue staining TSA workflow with one DARR stripping step is as in the following sequence:

1) Rehydrated tissue is placed into a pressure cooker that is filled with 1x Antigen Unmasking Solution, Citrate-Based (Vector Labs, Newark) and allowed to pressure cook for 1 minute at maximum pressure.

2) Endogenous peroxidase activity is blocked in tissue sections by applying Bloxall Endogenous Blocking Solution (Vector Labs, Newark) for 10 minutes.

3) Tissue sections are washed for 5 minutes with 1× PBS.

4) Non-specific binding is blocked in tissue sections by incubating with 2.5% Normal Horse Serum (2.5% NHS) for 30 minutes.

5) The block is tipped-off.

6) Primary binders, including primary antibodies, are applied to tissue sections. Primary binders are usually diluted in dilute animal serum to minimize off-target binding (i.e. 2.5% NHS). Each primary binder has a unique dilution amount for optimal staining.

7) Tissue sections are washed 3 × 2 minutes each with 1× TBST.

8) Tissue sections are incubated with HRP-labelled secondary antibodies in 1x PBS for 10-30 minutes (10 minutes using Akoya secondaries and 30 minutes for Vector secondaries).

9) Tissue sections are washed 3 × 2 minutes each with 1× TBST.

10) TSA substrates are diluted according to the vendor’s instructions and applied to tissue sections for 10 minutes.

11) Tissue sections are washed 3 × 2 minutes each with 1x TBST.

12) Detection agents are stripped off the tissue sections using DARR Reagent 1 for 15 minutes, then a rinse of 1× PBS followed by 15 minutes incubation of DARR Reagent 2.

13) DARR reagents are washed off for 5 minutes with 1x PBS.

14) Repeat staining can be performed by repeating this process from step 2. A different color of TSA substrate (dye) is used in step 10 for each cycle.

15) Tissue sections have 25 uL of VECTASHIELD Vibrance® Antifade Mounting Medium with DAPI (Vector Labs, Newark) applied followed by a coverslip.

16) Tissue sections are allowed to cure at room temperature for 30 minutes before imaging.

17) Imaging is performed on a Nikon fluorescent microscope.

### Protocol 7: Tissue Staining - Immunofluorescence With a DARR Stripping Step Followed By a Chase.

The tissue staining immunofluorescence workflow with one DARR stripping step, followed by a chase, is as in the following sequence:

1) Rehydrated tissue is placed into a pressure cooker that is filled with 1× Antigen Unmasking Solution, Citrate-Based (Vector Labs, Newark) and allowed to pressure cook for 1 minute at maximum pressure.

2) Non-specific binding in tissue sections is blocked by incubating with 2.5% Normal Horse Serum (2.5% NHS) for 30 minutes.

3) The block is tipped-off.

4) Primary binders, including primary antibodies, are applied to tissue sections. Primary binders are usually diluted in dilute animal serum to minimize off-target binding (i.e. 2.5% NHS). Each primary binder has a unique dilution amount for optimal staining.

5) Wash tissue sections for 5 minutes with 1× PBS.

6) Dye-labelled secondary antibodies, which are directed against the species of the primary antibody, are applied at 10 ug/mL diluted in 1x PBS or 2.5% NHS and incubate for 30 minutes.

7) Detection agents are stripped off the tissue sections using DARR Reagent 1 for 15 minutes, a rinse of 1× PBS followed by 15 minutes incubation of DARR Reagent 2.

8) DARR reagents are washed off for 5 minutes with 1× PBS.

9) A second dye-labelled secondary antibody, directed against the species of the primary antibody, are applied with a different dye or the same dye at 10 ug/mL diluted in 1× PBS or 2.5% NHS and incubate for 30 minutes. This secondary will probe for any unstripped primary binder and is called a “chase”.

10) Tissue sections are washed for 5 minutes with 1× PBS.

11) TrueView Autofluorescence Quenching Reagent (Vector Labs, Newark) is applied to the tissue sections for 5 minutes to minimize the autofluorescence.

12) Tissue sections are washed for 5 minutes with 1× PBS.

13) Tissue sections are mounted with 25 uL of VECTASHIELD Vibrance® Antifade Mounting Medium with DAPI (Vector Labs, Newark) and a coverslip to prevent fading and preserve the fluorescent signal.

14) Tissue sections are allowed to cure at room temperature for 30 minutes before imaging.

15) Imaging is performed on a Nikon fluorescent microscope.

### Protocol 8: Tissue Staining - TSA With a DARR Stripping Step Followed by a Chase.

The tissue staining TSA workflow with one DARR stripping step, followed by a chase, is as in the following sequence:

1) Rehydrated tissue is placed into a pressure cooker that is filled with 1× Antigen Unmasking Solution, Citrate-Based (Vector Labs, Newark) and allowed to pressure cook for 1 minute at maximum pressure.

2) Endogenous peroxidase activity is blocked in tissue sections by applying Bloxall Endogenous Blocking (Vector Labs, Newark) Solution for 10 minutes.

3) Tissue sections are washed for 5 minutes with 1× PBS.

4) Non-specific binding in tissue sections is blocked by incubating with 2.5% Normal Horse Serum (2.5% NHS) for 30 minutes.

5) The block is tipped-off.

6) Primary binders, including primary antibodies, are applied to tissue sections. Primary binders are usually diluted in dilute animal serum to minimize off-target binding (i.e. 2.5% NHS). Each primary binder has a unique dilution amount for optimal staining.

7) Tissue sections are washed 3 × 2 minutes each with 1× TBST.

8) Tissue sections are incubated with HRP-labelled secondary antibodies in 1 × PBS for 10-30 minutes (10 minutes using Akoya secondaries and 30 minutes for Vector secondaries).

9) Tissue sections are washed 3 × 2 minutes each with 1x TBST.

10) TSA substrates are diluted according to the vendor’s instructions and applied to tissue sections for 10 minutes.

11) Tissue sections are washed 3 × 2 minutes each with 1x TBST.

12) Detection agents are stripped off the tissue sections using DARR Reagent 1 for 15 minutes, a rinse of 1× PBS followed by 15 minutes incubation of DARR Reagent 2.

13) DARR reagents are washed off for 5 minutes with 1x PBS.

14) A second round of secondary antibody staining is applied to probe for remaining primary binders. HRP-labelled secondary antibodies are diluted in 1 × PBS and applied to the tissue sections for 10-30 minutes (10 minutes using Akoya (Akoya Biosciences, Boston, MA) secondaries and 30 minutes for Vector Labs secondaries).

15) Tissue sections are washed 3 × 2 minutes each with 1x TBST.

16) TSA substrates are diluted according to the vendor’s instructions and applied to tissue sections for 10 minutes. This chase step is usually done with dyes of a different color than the initial staining.

17) Tissue sections are washed 3 × 2 minutes each with 1x TBST.

18) Tissue sections have 25 uL of VECTASHIELD Vibrance® Antifade Mounting Medium with DAPI (Vector Labs, Newark) applied followed by a coverslip.

19) Tissue sections are allowed to cure at room temperature for 30 minutes before imaging.

20) Imaging is performed on a Nikon fluorescent microscope.

### Protocol 9 - Tissue Staining - Immunofluorescence With a Microwave Treatment (MWT) Stripping Step.

This protocol is identical to Protocol 5, except Protocol 5 steps 14 and 15 are replaced with the steps below:

14) Detection agents are stripped off the tissue sections using MWT. Slides are submerged in 1× Antigen Unmasking Solution, Citrate-Based (Vector Labs, Newark) and microwaved on high for 1 min, followed by microwaving at 20% power for 15 min.

15) Slides are washed for 5 minutes with 1× PBS.

### Protocol 10 - Tissue Staining- TSA With a Microwave Treatment (MWT) Stripping Step.

This protocol is identical to Protocol 6, except Protocol 6 steps 12 and 13 are replaced with the steps below:

12) Detection agents are stripped off the tissue sections using MWT. Slides are submerged in 1× Antigen Unmasking Solution, Citrate-Based (Vector Labs, Newark) and microwaved on high for 1 min, followed by microwaving at 20% power for 15 min.

13) Wash slides for 5 minutes with 1× PBS.

### Protocol 11 - Tissue Staining - Immunofluorescence With a Microwave Treatment MWT Stripping Step Followed by a Chase.

This protocol is identical to Protocol 7, except Protocol 7 steps 14 and 15 are replaced with the steps below:

14) Detection agents are stripped off the tissue sections using MWT. Slides are submerged in 1× Antigen Unmasking Solution, Citrate-Based (Vector Labs, Newark) and microwaved on high for 1 min, followed by microwaving at 20% power for 15 min.

15) Wash slides for 5 minutes with 1× PBS.

### Protocol 12 - Tissue Staining - TSA With a MWT Stripping Step Followed by a Chase.

This protocol is identical to Protocol 8, except Protocol 8 steps 12 and 13 are replaced with the steps below:

12) Detection agents are stripped off the tissue sections using MWT. Slides are submerged in 1× Antigen Unmasking Solution, Citrate-Based (Vector Labs, Newark) and microwaved on high for 1 min, followed by microwaving at 20% power for 15 min.

13) Wash slides for 5 minutes with 1× PBS.

### Protocol 13 - Tissue Staining by Hematoxylin.

The tissue staining by hematoxylin workflow is as in the following sequence:

1) Hematoxylin staining is performed by applying hematoxylin directly to the slides.

2) Sections are incubated with hematoxylin for 1-5 minutes.

3) Rinse sections with running tap water until rinse water is colorless.

4) Differentiate by dipping slides 10 times in acid rinse solution (2 ml glacial acetic acid plus 98 ml of deionized or distilled water) followed by 10 dips in tap water.

5) Incubate slides in appropriate bluing solution.

6) Mount sections in aqueous or permanent mounting media as per standard protocol.

### Protocol 14 - Tissue Staining by Hematoxylin and Eosin.

The tissue staining by hematoxylin workflow is as in the following sequence:

1) Apply adequate hematoxylin to completely cover tissue section and incubate for 5 minutes.

2) Rinse slides in 2 changes of distilled water (15 seconds each) to remove excess stain.

3) Apply adequate bluing reagent to completely cover tissue sections and incubate for 10-15 seconds.

4) Rinse the slides in 2 changes of distilled water (15 seconds each).

5) Dip slides in 100% ethanol (10 seconds) and blot excess off.

6) Apply adequate Eosin Y Solution to completely cover tissue sections and incubate for 2-3 minutes.

7) Rinse slides using 100% ethanol (10 seconds).

8) Dehydrate slides in 3 changes of 100% ethanol (1-2 minutes each).

6) Clear and coverslip.

### Additional Protocol Information

Common post-treatments to perform on tissue sections after stripping include blocking or re-blocking the tissue using any of the various blocking reagents that are commercially available, such as Animal-Free Block, Carbo-Free Block, Streptavidin/Biotin Block, animal serum, WestVision block, protein blocks, BSA, Salmon sperm DNA, genomic DNA, etc.

Non-aqueous versions of the process can be performed on slides that have been dehydrated (e.g. as in Protocol 2), subject to solubility and performance considerations in the alternative solvents. Non-aqueous solvents can also prove useful/beneficial for use in stripping surfaces other than tissues, such as, for example, sensors, metal surfaces, hydrogels, and the like.

Additional additives that can be added to improve or modulate performance in aqueous or non-aqueous compositions include but are not limited to, for example, surfactants, salts, anti-microbials, detergents, means of modifying pH (e.g. by adding acidic or basic components), buffering molecules, and the like.

### Example 2

### Detection Agent Removal With Ammonium Thiocyanate

### Method

Protocol 1 + Protocol 5 with ammonium thiocyanate as DARR Reagent 2 at various concentrations for 7 minutes. DARR Reagent 1: 10 mg/mL Tris-hydroxy propyl phosphine (Sigma: 777854-1g) in distilled water for 5 minutes. DARR Reagent 2: either 8 M, 4 M, or 2 M ammonium thiocyanate (Acros organics: 206505000) in distilled water.

Four tissue samples with a mix of rabbit antibodies (targeting ubiquitin, vimenten, and erlin2 antigens) bound to their relevant targets on the tissue and stained with subsequent DyLight™ 594 fluorescently-labeled anti-rabbit secondary antibodies were incubated with Reagent 1 at room temperature (25° C.) for 5 minutes. Reagent 1 was removed from the sample by tipping the reagent off the slide. 0.5 mL of Reagent 2 was then applied to the sample and allowed to incubate at room temperature for 7 minutes. Reagent 2 was then tipped off and another 0.5 mL of Reagent 2 was applied to the sample and incubated for 7 minutes. No washing was performed between the addition of Reagent 1 and Reagent 2 or between the first and second applications of Reagent 2. Tissue sample 1 was a positive control and had no detection agent removal performed. Tissue samples 2-4 had the detection agent removal process performed using 10 mg/mL Reagent 1 in combination with (2) 8 M Reagent 2, (3) 4 M Reagent 2, or (4) 2 M Reagent 2 for 7 minutes at room temperature, done twice. Reagent 2 was then washed from the samples using PBS and the tissue sample was stored in PBS at room temperature (25 C).

### Results

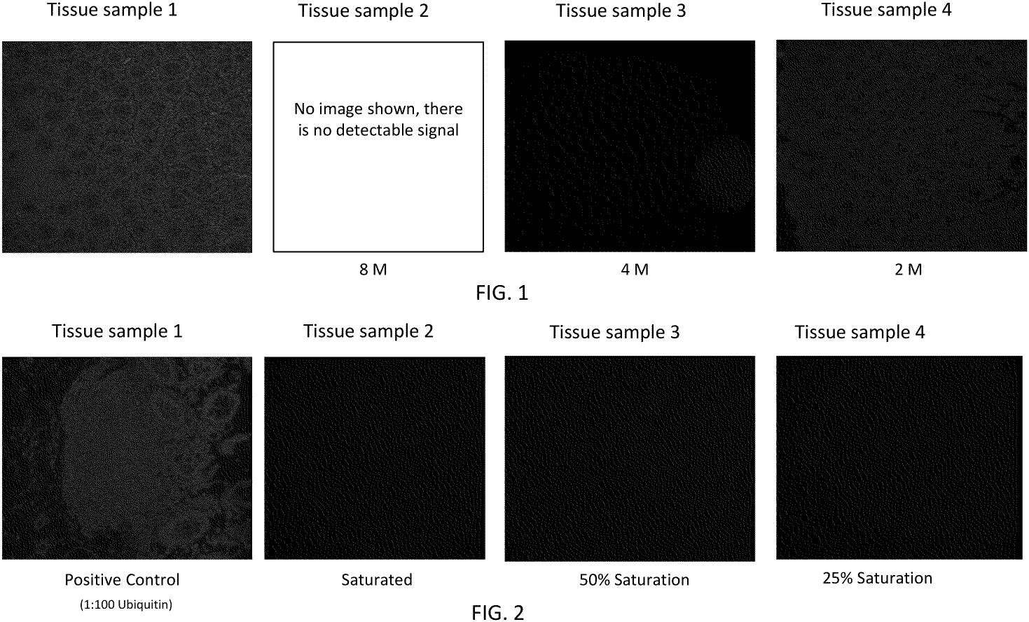

As shown in FIG. 1, positive staining (Tissue sample 1) is removed, with performance that correlates with DARR Reagent 2 concentrations. At 8 M (Tissue sample 2) and 4 M (Tissue sample 3), the signals are almost completely removed but when diluted to 2 M (Tissue sample 4), some signal remains. This indicates incomplete stripping when Reagent 2 is below 4 M ammonium thiocyanate.

Thus Reagent 2 could be diluted to 4 M and still effectively remove antibodies from the immunostained tissue samples. Images were generated using a Nikon camera with excitation at 594 nm to cause the DyLight™ 594 fluorophore to be excited and fluoresce.

### Example 3

### Detection Agent Removal With Guanidine Thiocyanate

### Method

Protocol 1 + Protocol 5 using guanidinium thiocyanate as DARR Reagent 2 at various concentrations. DARR Reagent 1: 10 mg/mL Tris-hydroxy propyl phosphine (Sigma: 777854-1 g) in distilled water for 10 minutes. DARR Reagent 2: either a saturated solution (~6 M), 50% saturation (~3 M), or 25% saturation (~1.5 M) guanidine thiocyanate in distilled water, incubated on the tissues for 7 minutes.

Four tissue samples with diluted rabbit anti-ubiquitin (1:100) bound to their relevant targets on the tissue and stained with subsequent DyLight™ 594 fluorescently-labeled anti-rabbit secondary antibodies were incubated with Reagent 1 at room temperature (25° C.) for 5 minutes. The tissue used in this experiment was FFPE human colon tissue. Sections were 5 um thick. Reagent 1 was removed from the sample by tipping the reagent off the slide. 0.5 mL of Reagent 2 was then applied to the sample and allowed to incubate at room temperature for 7 minutes. Reagent 2 was then tipped off and another 0.5 mL of Reagent 2 was applied to the sample and incubated for 7 minutes. No washing was performed between the addition of Reagent 1 and Reagent 2 or between the first and second applications of Reagent 2. Tissue sample 1 was a positive control and had no detection agent removal performed. Tissue samples 2-4 had the detection agent removal process performed using 10 mg/mL Reagent 1 in combination with (2) supernatant of saturated Reagent 2, (3) 50% saturated Reagent 2, or (4) 25% saturated Reagent 2 for 7 minutes at room temperature, done twice. Reagent 2 was then washed from the samples using PBS and the tissue sample was stored in PBS at room temperature (25 C).

### Results

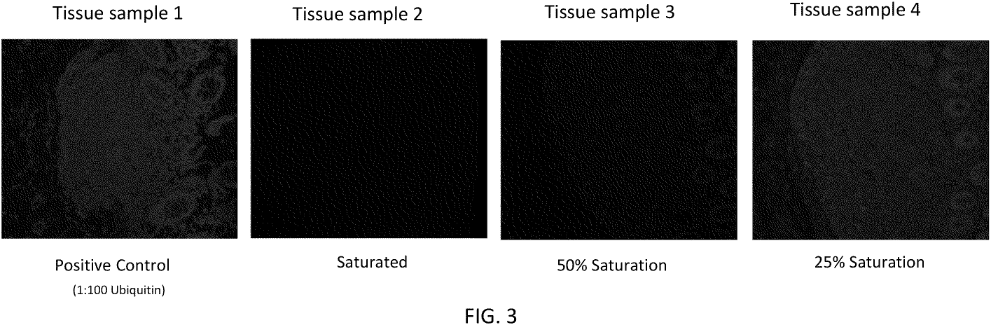

As shown in FIG. 2, positive staining (Tissue sample 1) is removed, with performance that correlates with DARR Reagent 2 concentrations. At saturated (Tissue sample 2), 50% saturated (Tissue sample 3), and 25% saturated (Tissue sample 4) solution concentrations the signals are almost completely removed.

The results (FIG. 2) show Reagent 2 could be diluted to 50% saturation and still effectively remove antibodies from the immunostained tissue samples. Images were generated using a Nikon camera with excitation at 594 nm to cause the DyLight™ 594 fluorophore to be excited and fluoresce.

### Example 4

### Detection Agent Removal With Sodium Perchlorate

### Method

Protocol 1 + Protocol 5 using sodium perchlorate as DARR Reagent 2 at various concentrations. DARR Reagent 1: 10 mg/mL Tris-hydroxy propyl phosphine (Sigma: 777854-1g) in distilled water for 10 minutes. DARR Reagent 2: either a saturated solution, a 50% saturated solution, or a 25% saturated solution of sodium perchlorate in distilled water, incubated on the tissues for 7 minutes.

Four tissue samples stained with diluted rabbit anti-ubiquitin (1:100) bound to their relevant targets on the tissue and stained with subsequent DyLight™ 594 fluorescently-labeled anti-rabbit secondary antibodies were incubated with Reagent 1 at room temperature (25° C.) for 5 minutes. The tissue used in this experiment was FFPE human colon tissue. Sections were 5 um thick. Reagent 1 was removed from the sample by tipping the reagent off the slide. 0.5 mL of Reagent 2 was then applied to the sample and allowed to incubate at room temperature for 7 minutes. Reagent 2 was then tipped off and another 0.5 mL of Reagent 2 was applied to the sample and incubated for 7 minutes. No washing was performed between the addition of Reagent 1 and Reagent 2 or between the first and second applications of Reagent 2. Tissue sample 1 was a positive control and had no detection agent removal performed. Tissue samples 2-4 had the detection agent removal process performed using 10 mg/mL Reagent 1 in combination with (2) supernatant of saturated Reagent 2, (3) 50% saturated Reagent 2, or (4) 25% saturated Reagent 2 for 7 minutes at room temperature, done twice. Reagent 2 was then washed from the samples using PBS and the tissue sample was stored in PBS at room temperature (25 C).

### Results

As shown in FIG. 3, positive staining (Tissue sample 1) is removed with performance that correlates with DARR Reagent 2 concentrations. At saturated solution concentrations (Tissue sample 2) the signals are almost completely removed. At 50% saturated (Tissue sample 3) and 25% saturated (Tissue sample 4) solution concentrations the signals can be seen indicating incomplete stripping.

The results (FIG. 3) show Reagent 2 could be diluted to half saturation (50%) and still effectively remove antibodies from the immunostained tissue samples. Images were generated using a Nikon camera with excitation at 594 nm to cause the DyLight™ 594 fluorophore to be excited and fluoresce.

### Example 5

### Autofluorescence

### Method

Autofluorescence can be present in FFPE tissues. To test for this, Protocol 1 + Protocol 12 was performed using Opal (Akoya) TSA reagents. Stained and imaging was done in the 570 channel for initial staining and the 520 channel for the chase staining.

### Results

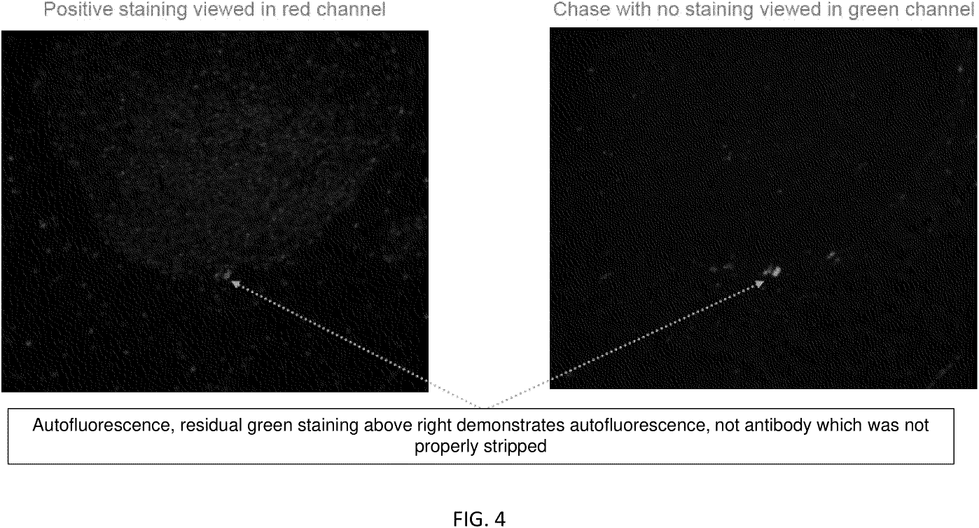

As shown in FIG. 4, autofluorescence can be seen as a specific signal that exists in multiple channels and is not related to detection agent staining. This data is representative of artifacts that can appear in chase experiments that look like residual staining from applied detection reagents but are not.

### Example 6

### Complete Stripping by Using Both Reagents in Combination

### Method

This system was run as a test method to demonstrate that the effectiveness of the present system relies on the presence of both reagents, namely the denaturant and the reductant, in combination.

FFPE tonsil or FFPE colon was stained using multiple primary antibodies in an IF workflow using Dylight 594 anti-Mouse IgG, followed by application of the DARR stripping reagent. Dylight 488 anti-Mouse IgG were then applied to bind to any unstripped mouse antibody.

Antibody removal performance was compared using a single reagent versus the combined reagent formulation. The residual primary reagent on the tissue was evaluated by applying an additional secondary antibody reagent. Reagent 1 was 5 mg/mL Tris-hydroxy propyl phosphine applied for 15 min, followed by rinsing with 1× PBS. Reagent 2 was 295 mg/mL guanidine thiocyanate for 15 min. Samples were then washed for 5 min in 1× PBS.

Protocol 1 was used followed by Protocol 5.

### Results

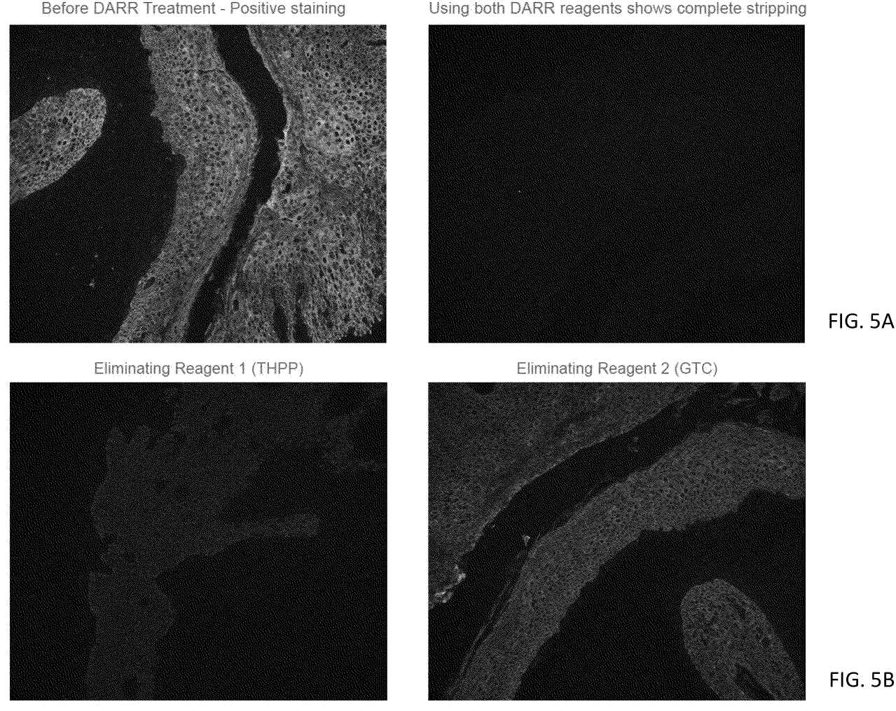

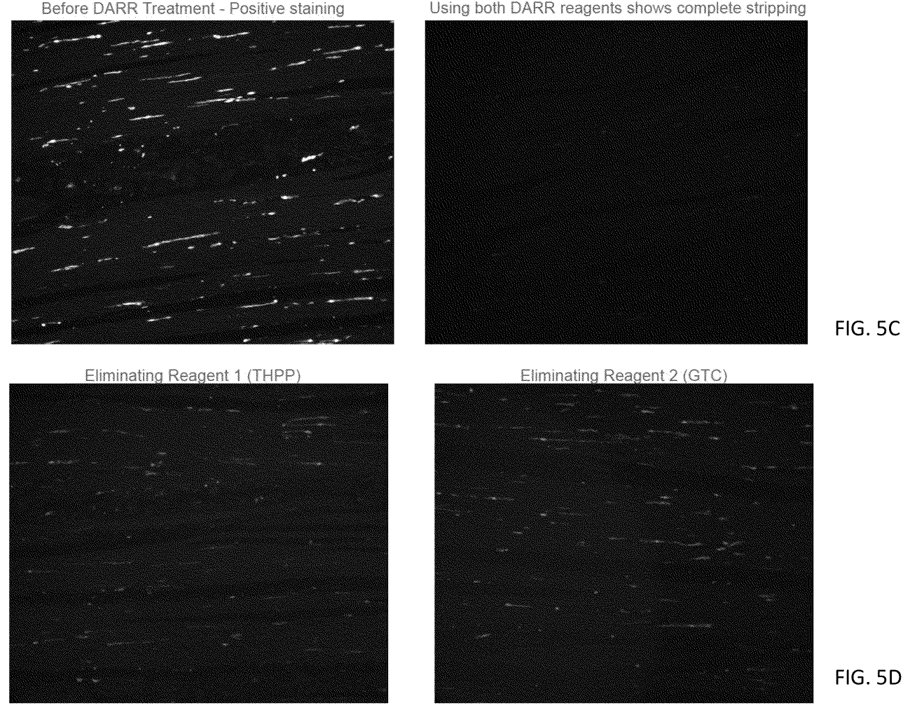

FIG. 5A and FIG. 5C show positive staining (left) and removal by DARR (right) demonstrating effective stripping of detection agents. FIG. 5B and FIG. 5D show stripping performance when one of the DARR reagents is omitted from the protocol; omission of either component yields suboptimal results and is not comparable to microwave stripping treatment.

Elimination of Reagent 1 (THPP; left) shows some signal remains while eliminating Reagent 2 (GTC; right) shows significant signal remains. GTC (chaotropic salt) omission had a bigger impact than omission of the THPP (reductant). Thus, it is clear that performance relies on the combination of both reagents.

### Example 7

### Verification of Complete Removal of Antibodies - Immunofluorescence

### Method

The Detection Agent Removal Reagent (DARR) composition and stripping procedure used: Reagent 1: 5 mg/mL tris(hydroxypropyl)phosphene (THPP) for 15 minutes at RT followed by PBS wash; and Reagent 2: 2.5 M Guanidine Thiocyanate (GTC), for 15 minutes at RT followed by PBS wash. Stripping is confirmed by performing chase experiment where a second round of secondary staining is performed after stripping to determine if residual primaries are still present.

The workflow involves first staining mouse primary antibody using Dylight 594 anti-Mouse IgG. The slide is then treated with DARR to remove detection reagents. Then, Dylight 488 anti-Mouse IgG is added to bind to any mouse primary antibodies that were not stripped. Images are obtained to determine if any residual signal remains in both the 488 and 594 fluorescent channels.

Protocol 1 was used, followed by Protocol 5.

### Results

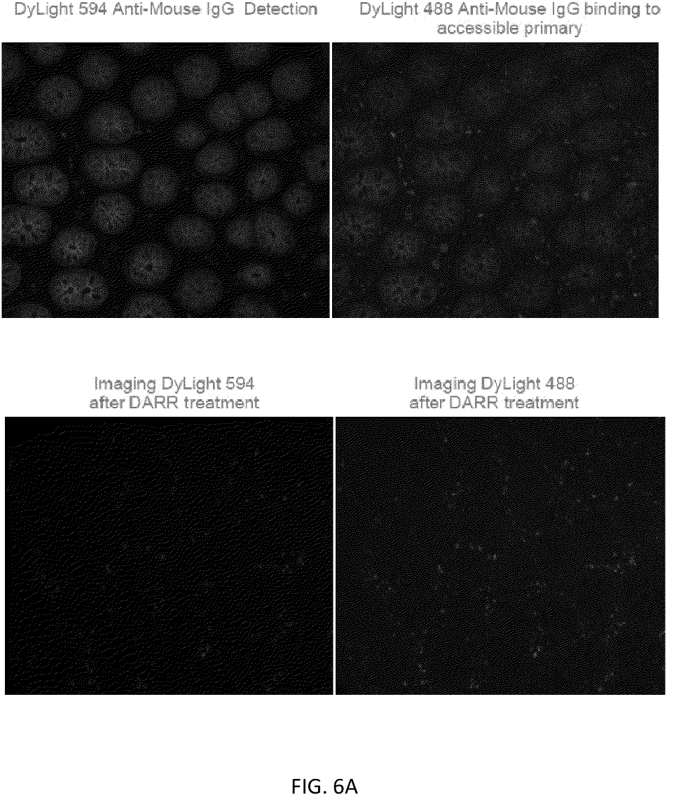

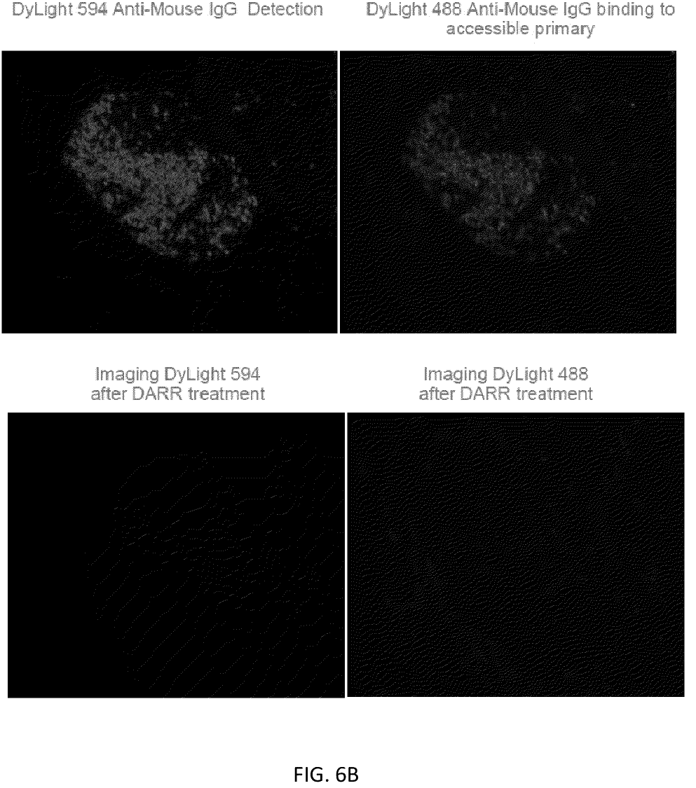

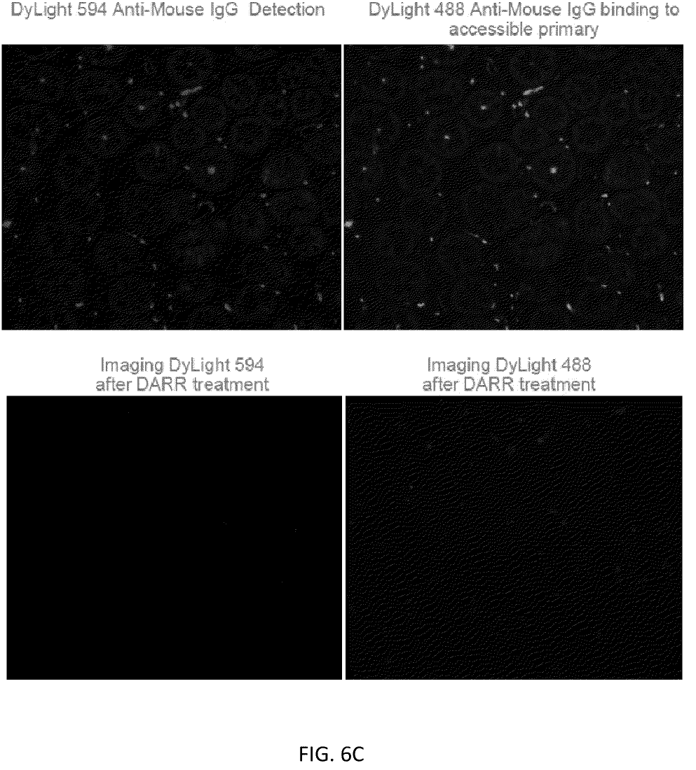

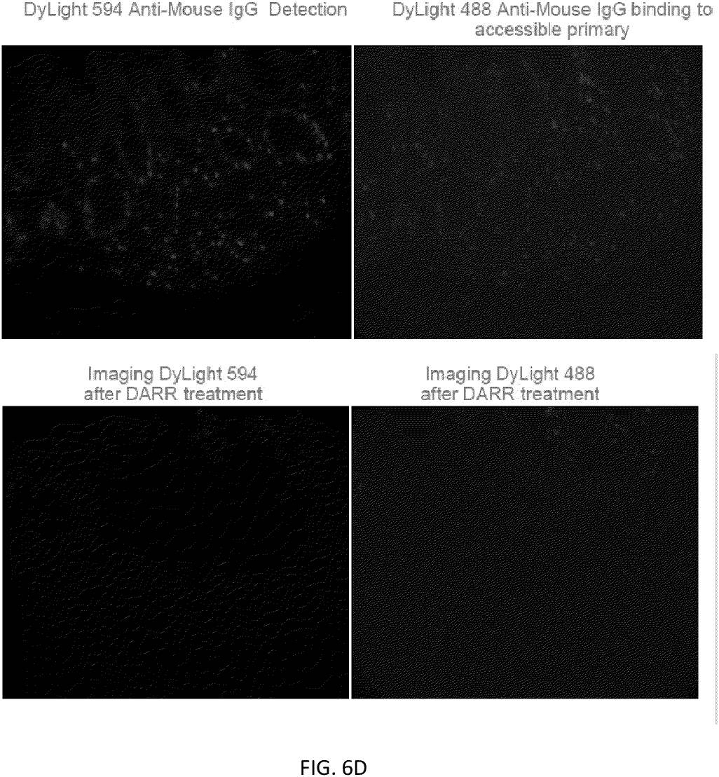

FIGS. 6A-D shows primary staining in colon tissue samples, giving positive signals on the top in both the 594 channel and the 488 channel. On the bottom is the imaging done in the same channels after DARR treatment. This demonstrates effective removal of detection reagents using the DARR method. This is shown for multiple primary antibodies used as the primary staining reagent.

Staining is not visible in either fluorescent channel after DARR treatment, thus indicating stripping of detection reagents.

### Example 8

### Verification of Complete Removal of Antibodies - Tyramide Signal Amplification (TSA) Method

Experiments were run to assess the performance of DARR vs microwave treatment (MWT) in the TSA workflow, which is an amplified detection system allowing greater signal intensities. Stripping of a panel of common cancer markers: anti-CD8, anti-CD68, anti-CD20, anti-AE1/AE3, anti-FoxP3, and anti-Ki67p was evaluated in the TSA workflow. The fluorophores are covalently bound to tissue sections.

MWT was used as the gold standard for stripping performance, such that DARR had to be comparable to MWT. DARR treatment should ideally: (1) have minimal impact on morphology/antigenicity, and (2) remove all antibodies from the specimen. Further, DARR treatment should ideally not quench covalently bound fluorophores.

The workflow involves first detecting mouse primary antibodies using HRP anti-Mouse IgG and Opal tyramide dyes. Detection reagents are then removed using DARR. A chase of a second round of HRP anti-Mouse IgG as the secondary is then applied, followed by Opal tyramide using a different dye to evaluate stripping efficiency.

Protocol 1 was used, followed by either Protocol 8 or Protocol 12. Protocol 8 was used for the DARR column of images on the left and Protocol 12 was used for MWT column of images on the right.

### Results

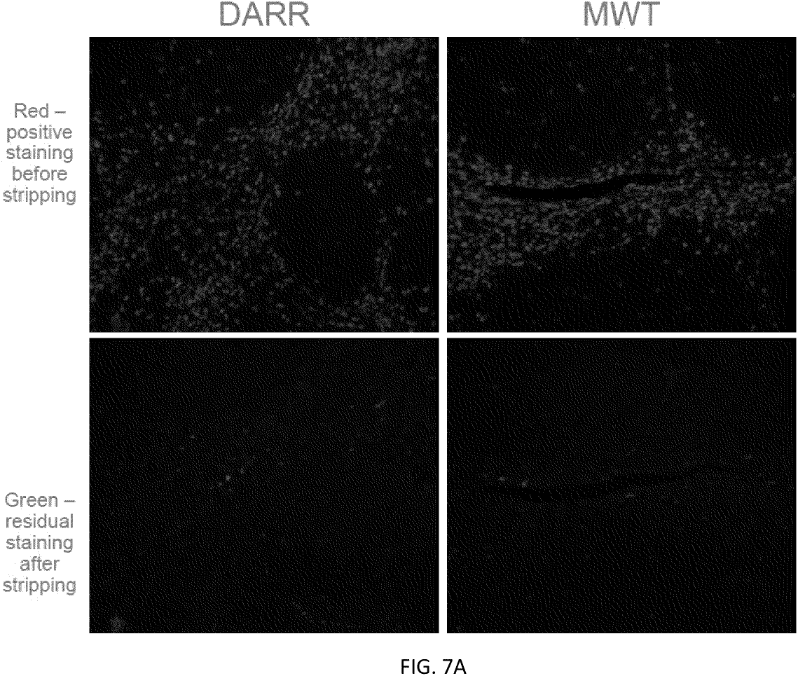

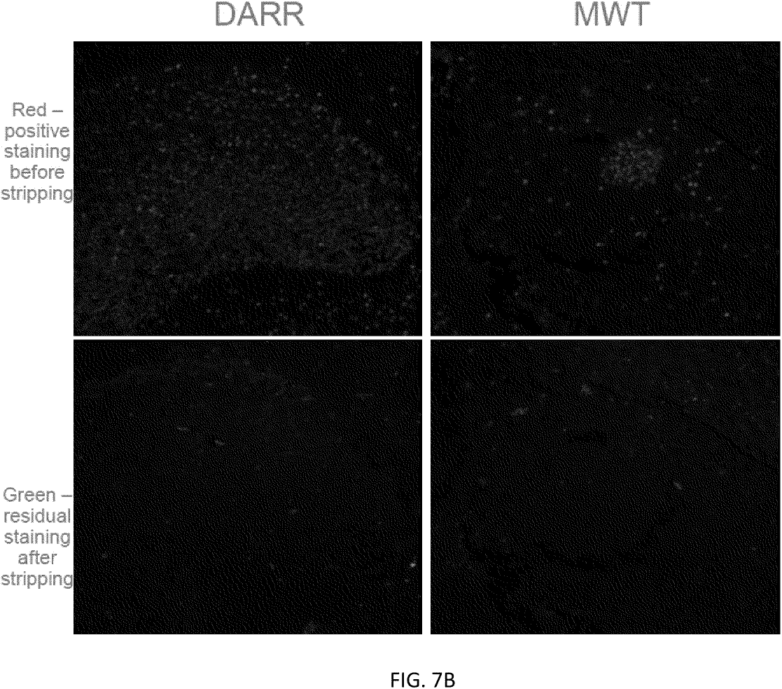

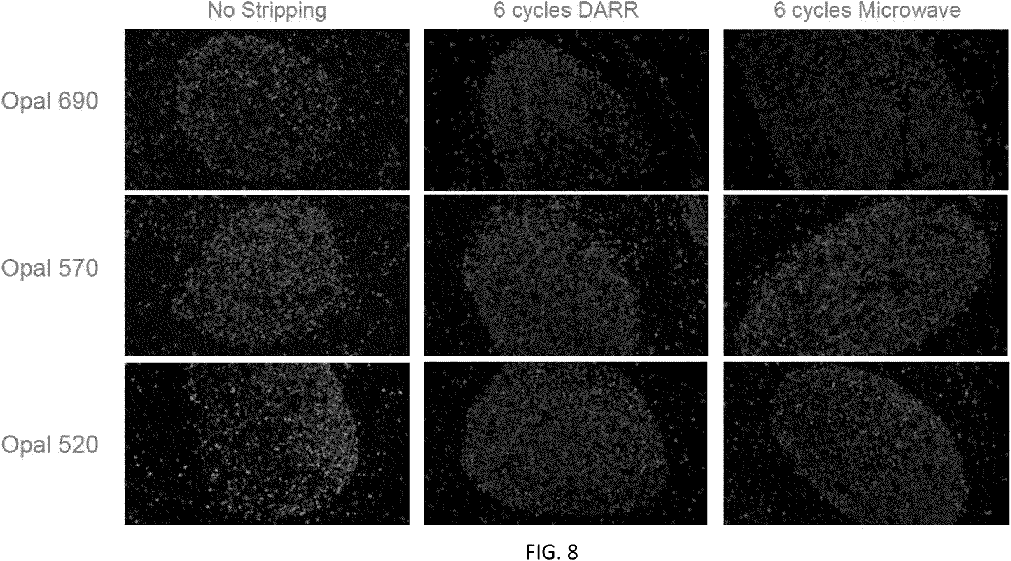

As shown in FIGS. 7A-B, DARR protocols are effective at removing detection reagents and are equivalent or superior in performance to MWT. Data are shown for two specific primary antibodies.

Both anti-CD8 and anti-Ki67p are stripped well with both DARR and MWT, with the DARR stripping being equivalent to that from MWT. Thus, DARR and MWT look equivalent in the TSA workflow, using a panel of relevant primary antibodies.

### Example 9

### DARR on Tyramide Dye Intensity

### Method

This workflow assesses DARR’s impact on tyramide dyes attached to the surface (using data for Opal dyes from AKOYA). In this process, DARR will be in direct contact with dyes already deposited and could potentially interfere/reduce their fluorescence.

Zero and six cycles of DARR or MWT were compared after TSA staining using 3 Opal dyes (Opal 520, 570, and 690), tested on FFPE tonsil with anti-Ki67p. Opal 520 and 570 are listed by AKOYA as “at risk” of sensitivity loss after microwave stripping.

Protocol 1 was used, followed by the following steps:

1) Rehydrated tissue is placed into a pressure cooker that is filled with 1× Antigen Unmasking Solution, Citrate-Based (Vector Labs, Newark) and allowed to pressure cook for 1 minute at maximum pressure.

2) Endogenous peroxidase activity in tissue sections is blocked by applying Bloxall Endogenous Blocking Solution (Vector Labs, Newark) for 10 minutes.

3) Tissue sections are washed for 5 minutes with 1× PBS.

4) Non-specific binding in tissue sections is blocked by incubating with 2.5% Normal Horse Serum (2.5% NHS) for 30 minutes.

5) The block is tipped-off.

6) Primary binders, including primary antibodies, are applied to tissue sections. Primary binders are usually diluted in dilute animal serum to minimize off-target binding (i.e. 2.5% NHS). Each primary binder has a unique dilution amount for optimal staining.

7) Tissue sections are washed 3 × 2 minutes each with 1× TBST.

8) Tissue sections are incubated with HRP-labelled secondary antibodies in 1× PBS for 10-30 minutes (10 minutes using Akoya secondaries and 30 minutes for Vector secondaries).

9) Tissue sections are washed 3 × 2 minutes each with 1× TBST.

10) TSA substrates are diluted according to the vendor’s instructions and applied to tissue sections for 10 minutes.

11) Tissue sections are washed 3 × 2 minutes each with 1× TBST.

12) Stripping is then either not performed as a control (column 1), performed using DARR reagents 6 times in a row (Column 2; Reagent 1 (5 mg/mL THPP) for 15 minutes, a rinse of 1× PBS followed by 15 minutes incubation of DARR Reagent 2 (295 mg/mL GTC)), or by MWT 6 times in a row (Column 3; Slides are submerged in 1× Antigen Unmasking Solution, Citrate-Based (Vector Labs, Newark) and microwaved on high for 1 min, followed by microwaving at 20% power for 15 min).

13) Stripping reagents are washed off for 5 minutes with 1× PBS.

14) Tissue sections have 25 uL of VECTASHIELD Vibrance® Antifade Mounting Medium with DAPI (Vector Labs, Newark) applied followed by a coverslip.

15) Tissue sections are allowed to cure at room temperature for 30 minutes before imaging.

16) Imaging is performed on a Nikon fluorescent microscope.

### Results

The data shown in FIG. 8 demonstrate that the DARR and MWT protocols on TSA stained tissues up to 6 cycles of stripping show little to no impact on the TSA dyes already attached to the tissue. It is clear from FIG. 8 that DARR methods and MWT are equivalent in terms of their impact on dyes.

### Example 10

### Comparing DARR With Biocare Denaturing Solution

### Method

DARR reagents were compared to Biocare’s Denaturing Solution using TSA and IF. TSA was tested on tonsil tissue with anti-Ki67p and anti-AE1/AE3 with Opal 520 primary staining and 570 chase (repeat secondary staining after stripping). IF was tested on tonsil and colon tissue with anti-Ki67p, anti-AE1/AE3 and anti-S100p primary staining and chase detection. One set was stripped with DARR and the other was stripped with Biocare Denaturing Solutionat its most concentrated formulation (1:1) for 5 minutes.

The workflow to evaluate the Biocare Denaturing Solution involved first detecting mouse antibody + HRP anti-Mouse IgG and Opal tyramide. Detection regents are then removed using DARR or Biocare Denaturing Solution. A chase of HRP anti-Mouse IgG is then applied, followed by Opal tyramide in a different channel to evaluate stripping efficiency.

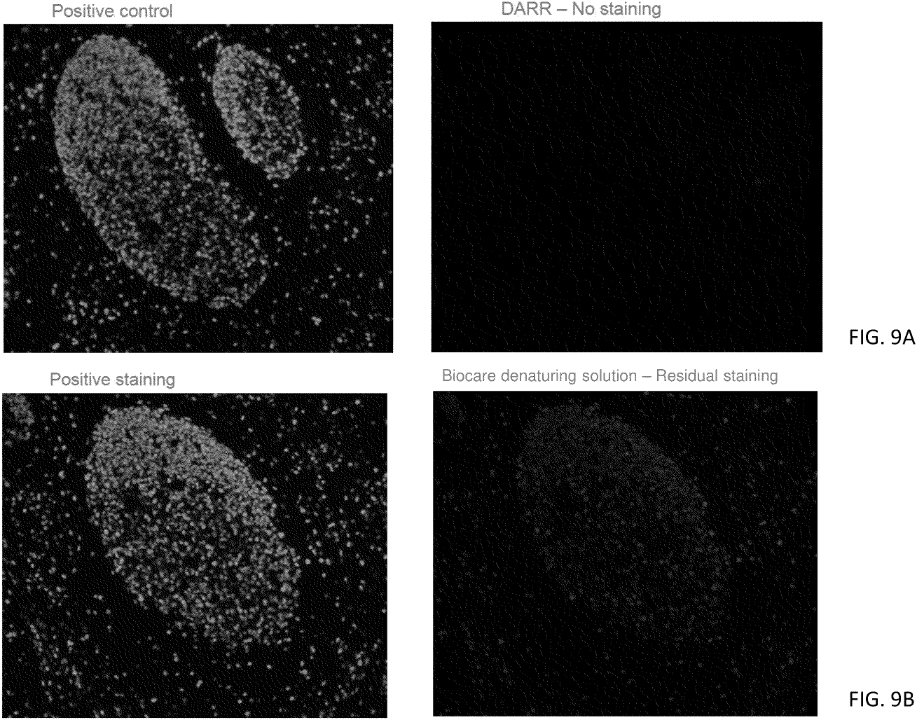

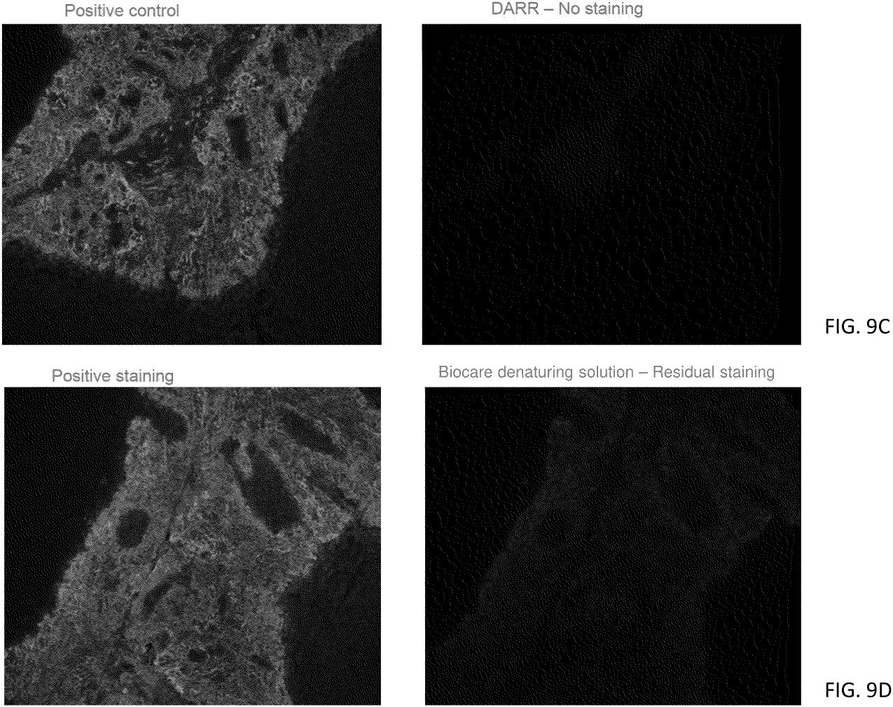

For the TSA analysis, Protocol 1 was used, followed by Protocol 8. For FIGS. 9B and 9D, showing Biocare Denaturing Solution performance, step 12 of Protocol 8 was replaced with the following step: 12) Biocare Denaturing Solution reagents 1 and 2 were mixed 1:1 right before being applied to the tissue sections for 5 minutes.

For the IF analysis, Protocol 1 was followed by Protocol 7. For the Biocare Denaturing Solution performance, step 14 of Protocol 7 was replaced with the following step: 14) Biocare Denaturing Solution reagents 1 and 2 were mixed 1:1 right before being applied to the tissue sections for 5 minutes.

### Results

As shown in FIGS. 9A-D, DARR reagents remove detection reagents well in TSA workflows (FIG. 9A and FIG. 9C) while Biocare Denaturing Solution’s performance is not as good (FIG. 9B and FIG. 9D).

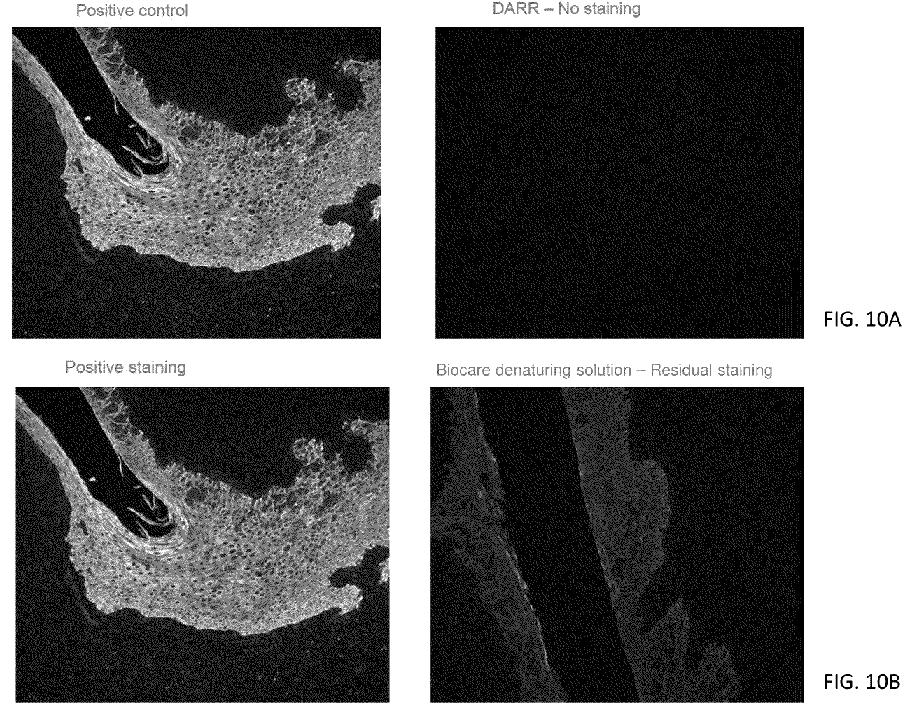

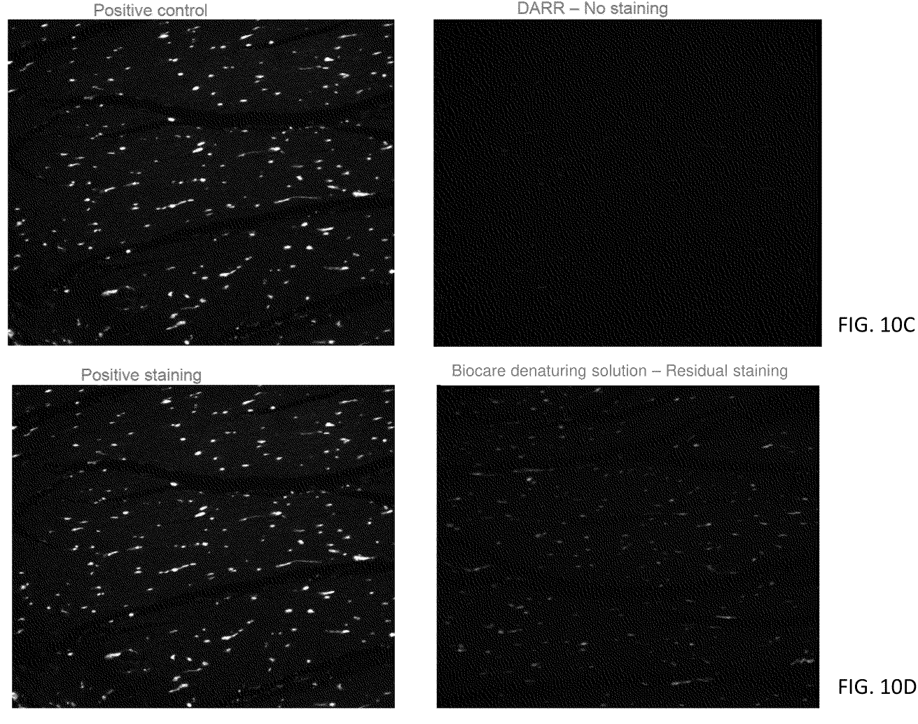

As shown in FIGS. 10A-D, DARR reagents remove detection reagents well in IF workflows (FIG. 10A and FIG. 10C) while Biocare Denaturing Solution’s performance is not as good (FIG. 10B and FIG. 10D).

FIGS. 9A-D shows that DARR strips anti-Ki67p and anti-AE1/AE3 well in the TSA workflow, whereas the Biocare Denaturing Solution does not completely strip either detection reagent. Meanwhile, FIGS. 10A-D shows that DARR strips anti-AE1/AE3 and anti-S100p in the IF workflow, whereas the Biocare reagent does not completely strip either detection reagent. The Biocare reagent had notable residual staining in all assays.

### Example 11

### Antigenicity Between DARR and MWT in the TSA Workflow

### Method

The antigenicity of markers within tissues was assessed before and after stripping using the TSA detection system, after running zero and six cycles of DARR or microwave treatment. Following treatments, tissue sections were stained with various primaries which were detected using the TSA workflow with Opal 690. Staining intensities before and after stripping treatments were then compared.

Protocol 1 was used, followed by Protocol 4 with 0 or 6 cycles of stripping inserted between step 1 and 2 of Protocol 4. Stripping is either not performed as a control (column 1), performed using DARR reagents 6 times in a row (Column 2; Reagent 1 (5 mg/mL THPP) for 15 minutes, a rinse of 1× PBS followed by 15 minutes incubation of DARR reagent 2 (295 mg/mL GTC)), or by MWT 6 times in a row (Column 3; Slides are submerged in 1× Antigen Unmasking Solution, Citrate-Based and microwaved on high for 1 min, followed by microwaving at 20% power for 15 min).

### Results

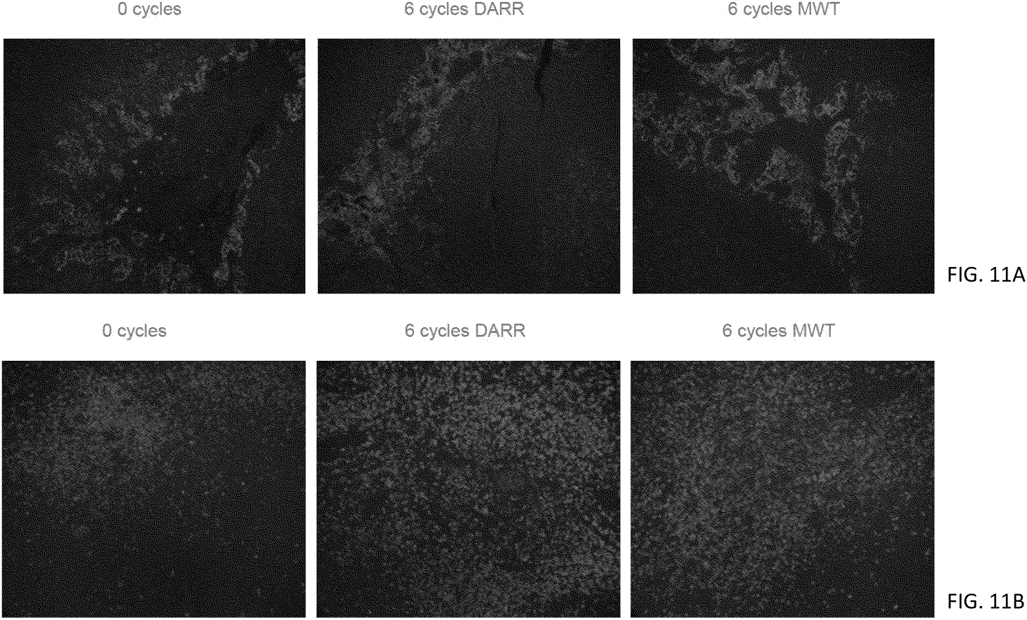

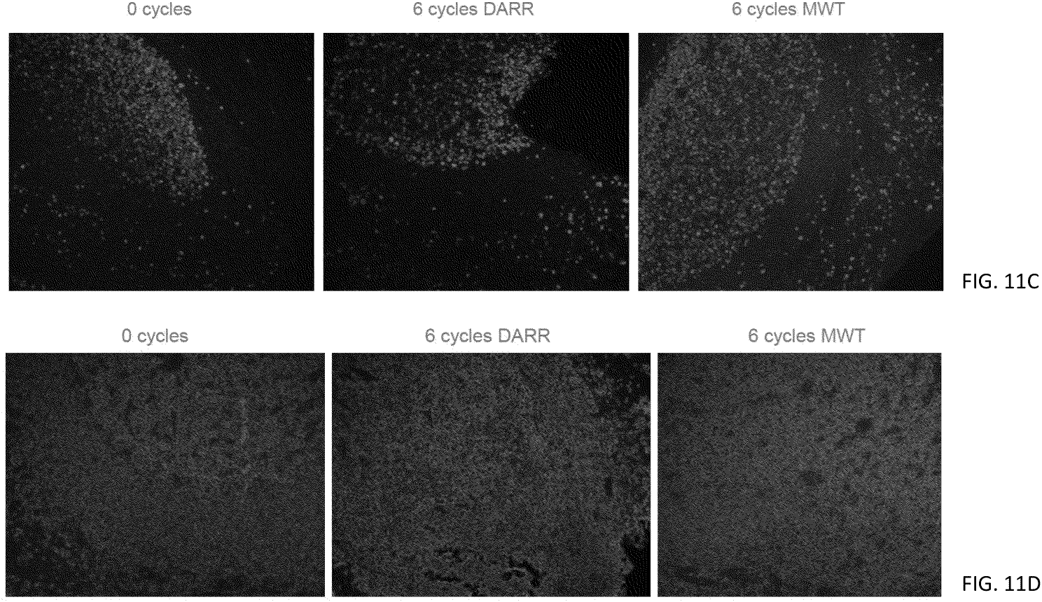

As shown in FIGS. 11A-D, in the TSA workflow, antigenicity of specific markers (anti-PDL-1, anti-CD3, anti-Ki67p, and anti-CD20), is not changed by treating tissues with 6 cycles of either DARR or MWT before staining as compared to no stripping being performed.

Antigenicity is thus not impacted by up to 6 cycles of DARR using the fluorescent TSA workflow. Results are comparable to microwave treatment (MWT).

### Example 12

### Antigenicity of DARR in the IF Workflow

### Method

The antigenicity of markers in tissues was assessed before and after DARR treatment using an IF detection system. After zero, one and six cycles of DARR, samples were stained using IF workflows. Various primaries were run and stained using 594 nm dye-labelled secondaries as the IF detection modality.

Protocol 1 was used followed by Protocol 3 with 0, 1 or 6 cycles of DARR stripping inserted between step 1 and 2 of Protocol 3. Stripping is Reagent 1 (5 mg/mL THPP) for 15 minutes, a rinse of 1× PBS followed by 15 minutes incubation of DARR Reagent 2 (295 mg/mL GTC). Stripping is either not performed as a control (column 1), performed once using DARR reagents (Column 2) or 6 times using DARR reagents (Column 3).

### Results

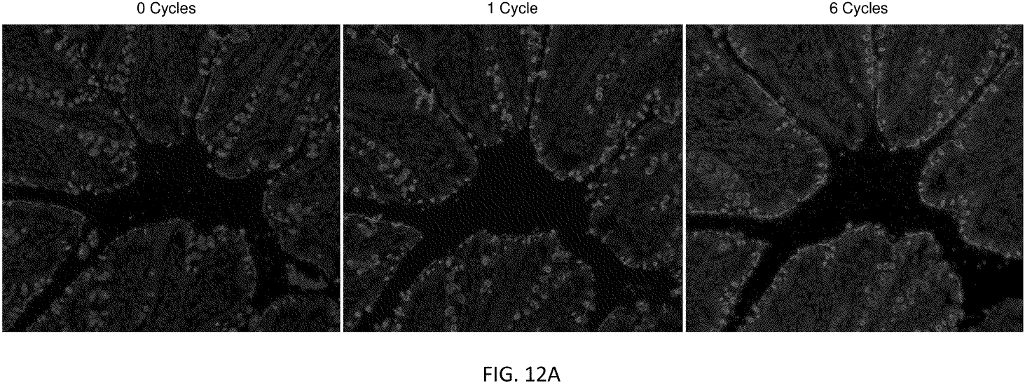

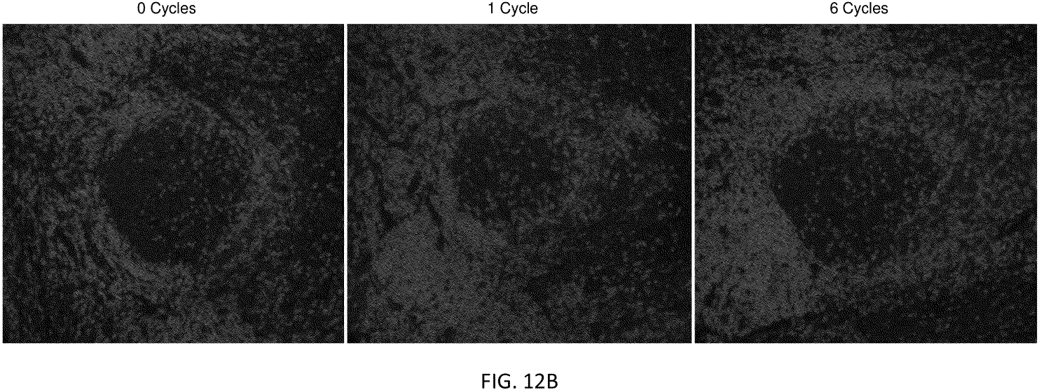

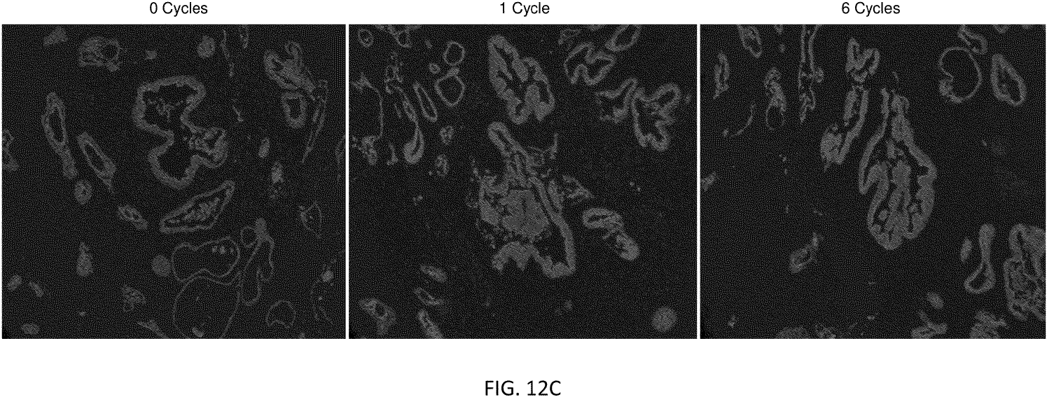

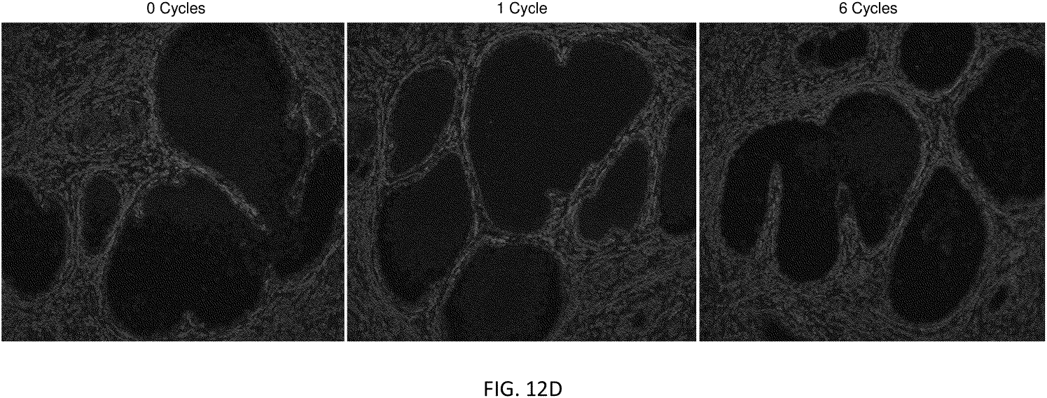

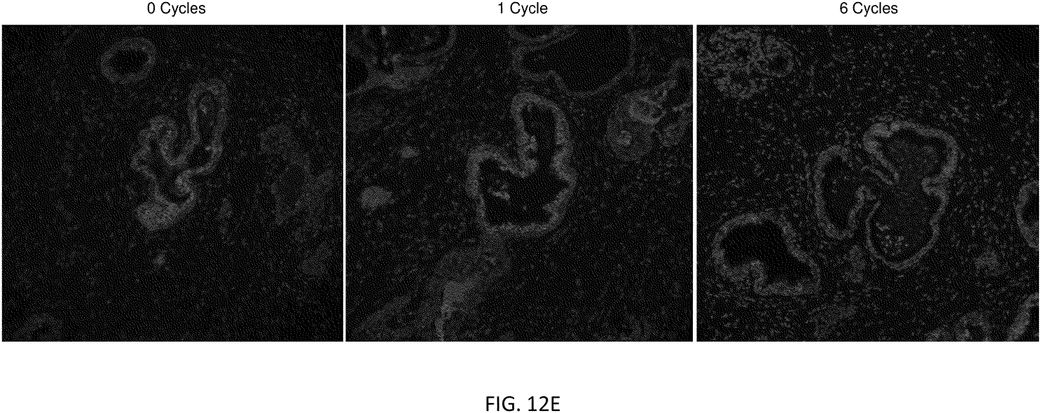

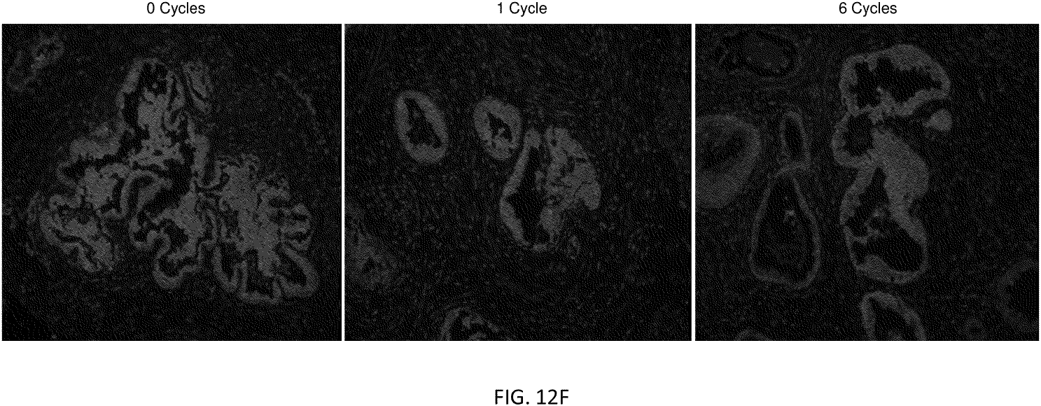

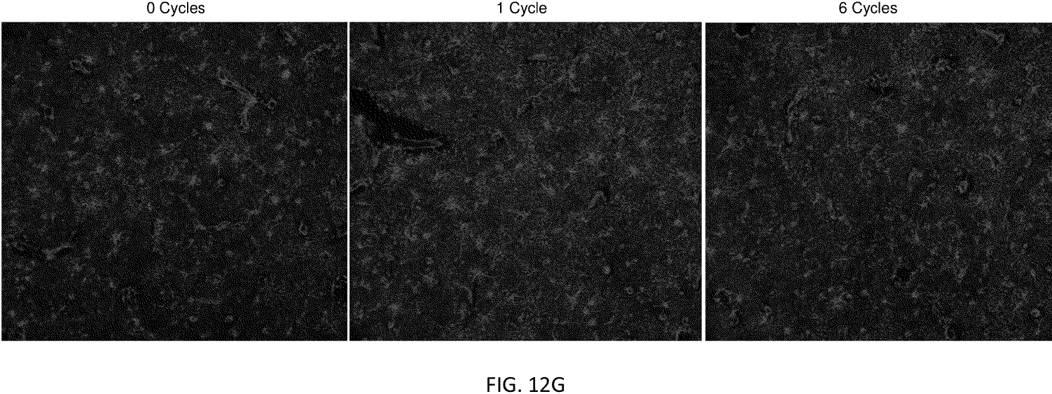

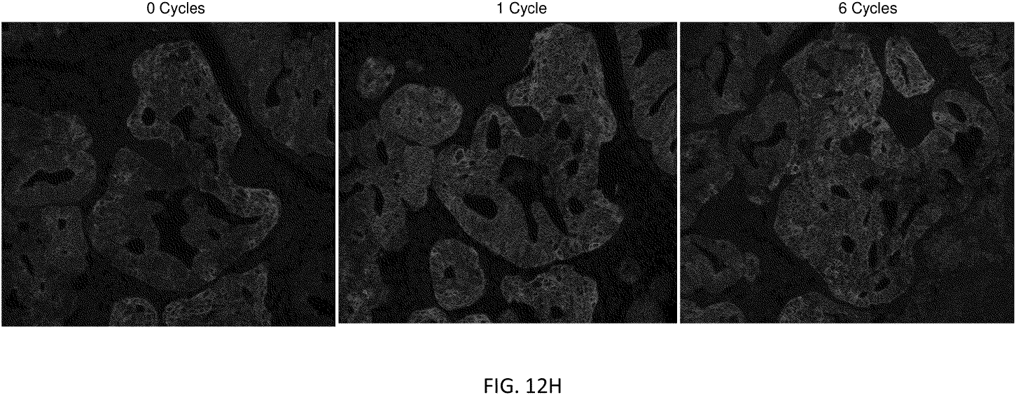

As shown in FIGS. 12A-H, in the IF workflow, antigenicity of specific markers in colon tissue (anti-CA19-9), tonsil tissue (anti-CD3), prostate tissue (anti-CD11, anti-SMA, anti-PAP, anti-PSA), brain tissue (anti-GFAP), and colon carcinoma (anti-C11) is not changed by treating tissues with 1 or 6 cycles of DARR treatment before staining as compared to no stripping being performed. Antigenicity of tissue sections shows minimal impact after 6 rounds of DARR treatment on multiple tissue types and detecting a variety of antigens.

### Example 13

### Tissue Damage After 6 Cycles of DARR or Pressure Cooking

### Method

The impact to tissue between DARRor pressure cooking was then assessed. First, FFPE tissue sections from multiple tissue types were deparaffinized and rehydrated. An antigen retrieval procedure was performed using a pressure cooker with citrate buffer. Tissue sections were subjected to one of three procedures for stripping: 1) no stripping; 2) 6 cycles of stripping using pressure cooker / citrate buffer; 3) 6 cycles of DARR. Slides were then stained with hematoxlin counterstain to evaluate tissue/nuclear morphology.

Various tissues were subjected to 6 cycles of pressure cooking-based stripping (rehydrated tissue is placed into a pressure cooker that is filled with 1× Antigen Unmasking Solution, Citrate-Based and allowed to pressure cook for 1 minute at maximum pressure) or 6 cycles of DARR stripping (DARR Reagent 1 (5 mg/mL THPP) for 15 minutes, a rinse of 1× PBS followed by 15 minutes incubation of DARR Reagent 2 (295 mg/mL GTC)). Protocol 13 was then used to stain the sections and Protocol 2 was used to dehydrate and mount the slides.

### Results

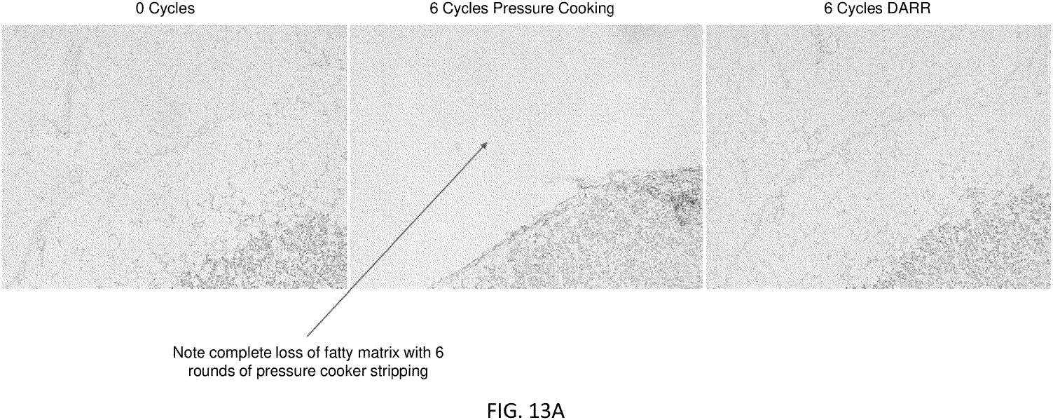

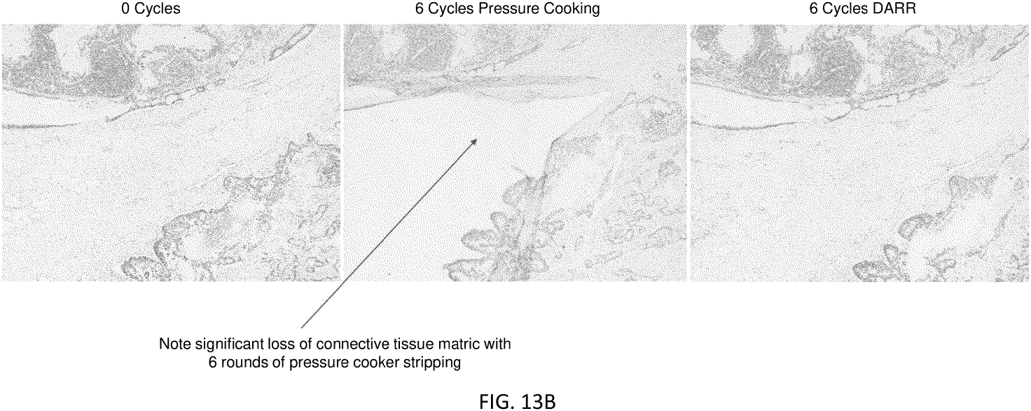

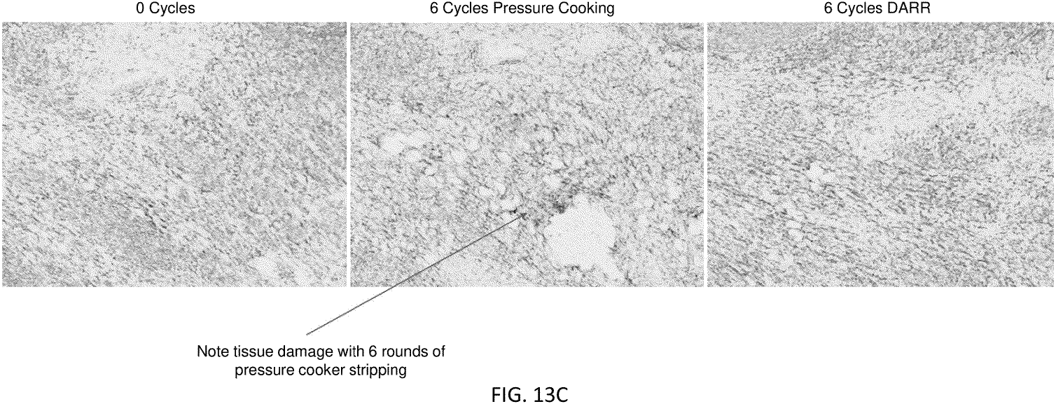

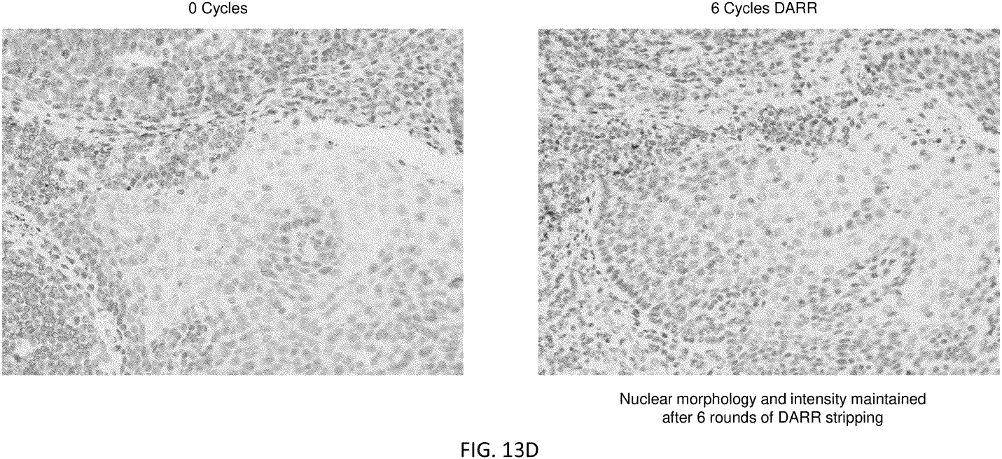

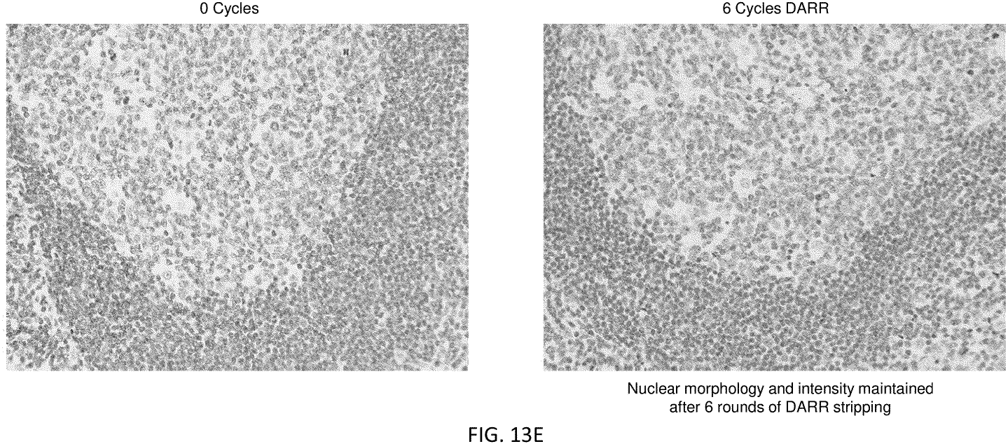

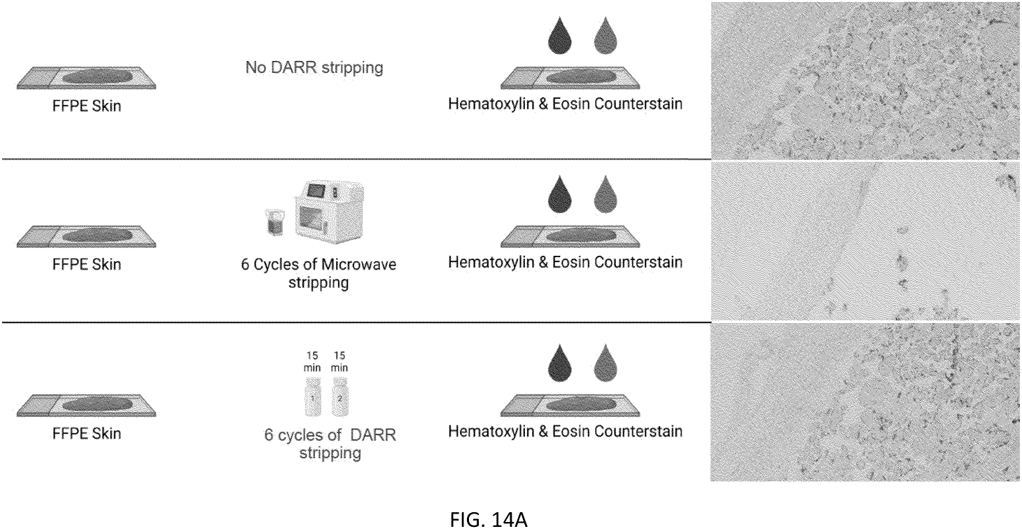

As shown in FIGS. 13A-E, tissue and nuclear integrity are maintained through 6 cycles of DARR while being heavily compromised by pressure cooking-based stripping. Unlike pressure cooker treatment, DARR was found to preserve tissue integrity in breast cancer tissue (FIG. 13A), colon cancer tissue (FIG. 13B), tonsil tissue (FIG. 13C, FIG. 13D, FIG. 13E); in particular, there is a complete loss of fatty matrix in breast cancer tissue, a significant loss of connective tissue matrix in colon cancer tissue, and general tissue damage in tonsil tissue with 6 rounds of pressure cooker stripping. In contrast, DARR preserves tissue and nuclear integrity in all types of tissues after 6 stripping cycles. The nuclear morphology and intensity are clearly maintained after 6 rounds of DARR stripping, as can be clearly seen at 20× amplification (FIG. 13D, FIG. 13E).

### Example 14

### Tissue Integrity After 6 Cycles of DARR or MWT

### Method

Various tissues were subjected to 6 cycles of MWT (slides are submerged in 1× Antigen Unmasking Solution, Citrate-Based and microwaved on high for 1 min, followed by microwaving at 20% power for 15 min) or 6 cycles of DARR stripping (DARR Reagent 1 (5 mg/mL THPP) for 15 minutes, a rinse of 1× PBS followed by 15 minutes incubation of DARR Reagent 2 (295 mg/mL GTC)). Protocol 14 was then used to stain the sections and Protocol 2 was used to dehydrate and mount the slides.

Specifically, FFPE skin tissue and FFPE breast carcinoma tissue samples were mounted and then subject to 6 cycles of DARR or MWT stripping, followed by hematoxylin and eosin counterstain.

### Results

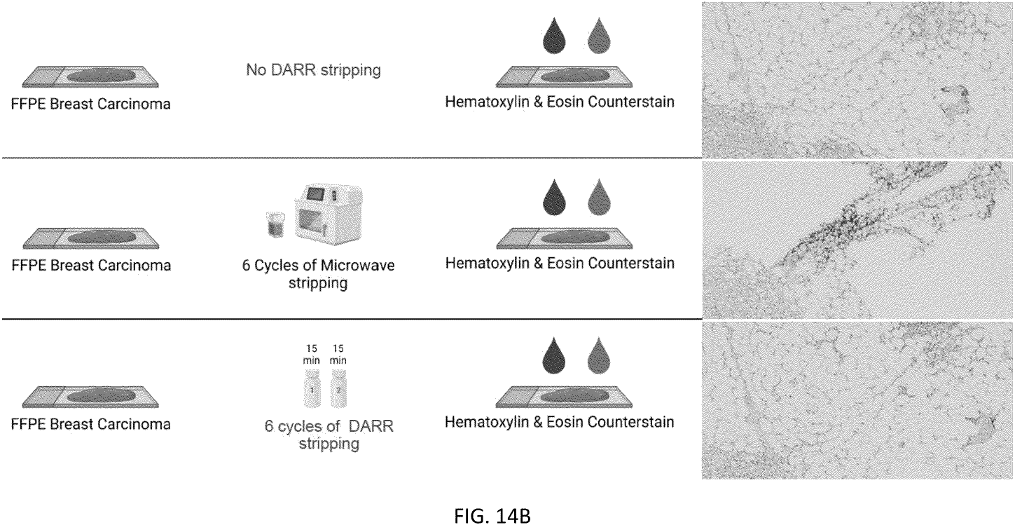

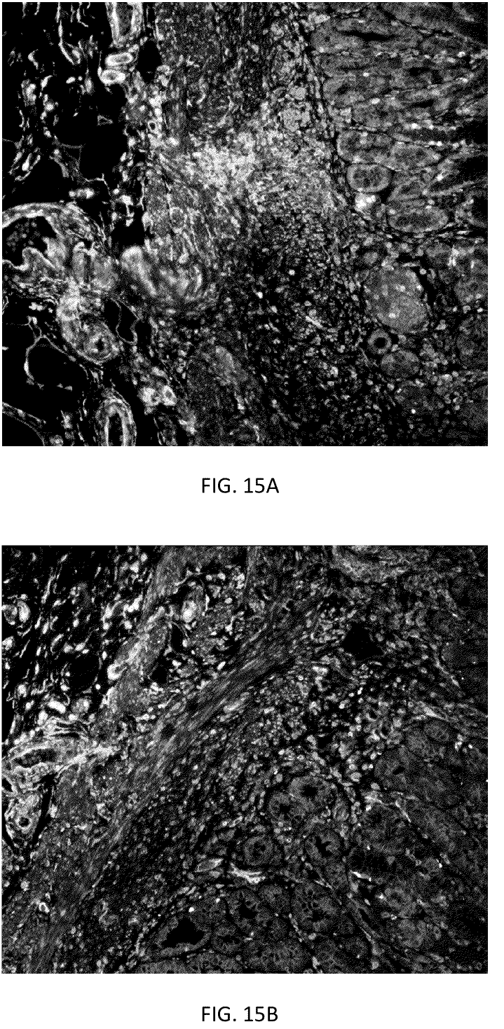

As shown in FIGS. 14A-B, tissue integrity is maintained through 6 cycles of DARR while being heavily compromised by MWT based stripping.

In view of the severe and negative impact pressure cooking has on tissue morphology, and the notable damage caused by microwave treatment, DARR shows a significant advantage in terms of maintaining tissue morphology as compared to either method, which would not be appropriate for any process involving multiple stripping steps and/or methods where the integrity of the tissue is desired to be preserved.

### Example 15

### Multiplex Imaging in the IF Workflow

### Method

A 6-plex image was obtained of FFPE stomach tissue. All mouse primaries were analyzed, i.e. anti-CD20, anti-CD34, anti-DES, anti-AE1/AE3, anti-Ki67p, and anti-VIM. Protocol 1 was used, followed by Protocol 5, with 6 rounds of stripping/staining.

### Results

As shown in FIG. 15, different combinations of 6 distinct markers, namely anti-CD20, anti-CD34, anti-DES, anti-AE1/AE3, anti-Ki67p, anti-VIM, and DAPI (FIG. 15A) and anti-CD20, anti-CD34, anti-DES, anti-AE1/AE3, anti-CD3, anti-VIM, and DAPI (FIG. 15B) can be clearly seen even after 6 rounds of stripping. In full color imaging, the CD20 shows as red, CD34 as yellow, DES as cyan, AE1/AE3 as magenta, Ki67p or CD3 as gray, VIM as green, and DAPI as blue. Thus, DARR allows at least 6 primary binding agents and one DAPI staining to be aligned, merged and visualized using DARR treatment. Certainly, additional binding agents can be incorporated and envisaged.

### Example 16

### DARR Treatment With Reagents in Reverse Order

### Method

DARR performance was evaluated when the DARR components are used in reverse order (GTC first and then THPP). FFPE tonsil and colon tissues were stained with anti-AE1/AE3, anti-Ki67p, and anti-S100p, using Protocol 1 followed by Protocol 7. When reverse order is tested, replace step 7 in Protocol 7 with: 7) Detection agents are stripped off the tissue sections using DARR Reagent 2 for 15 minutes, a rinse of 1× PBS followed by 15 min incubation of DARR Reagent 1.

### Results

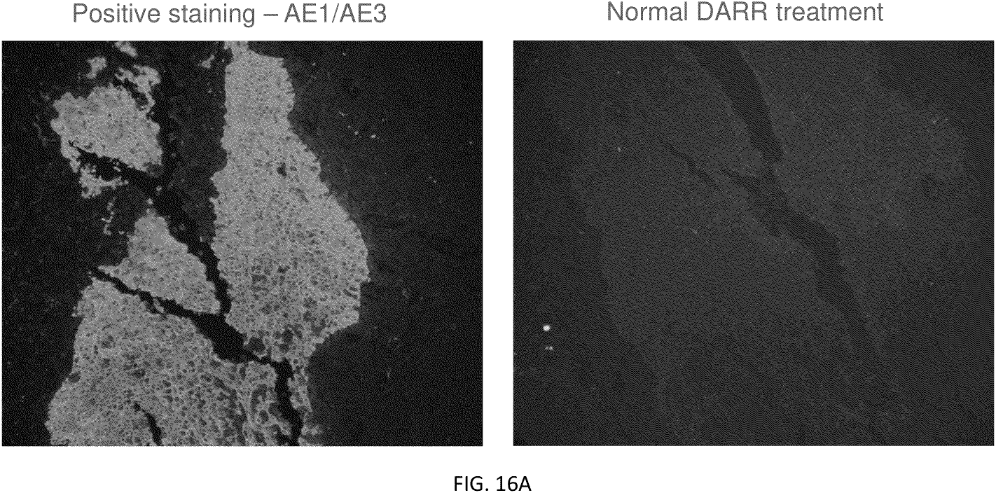

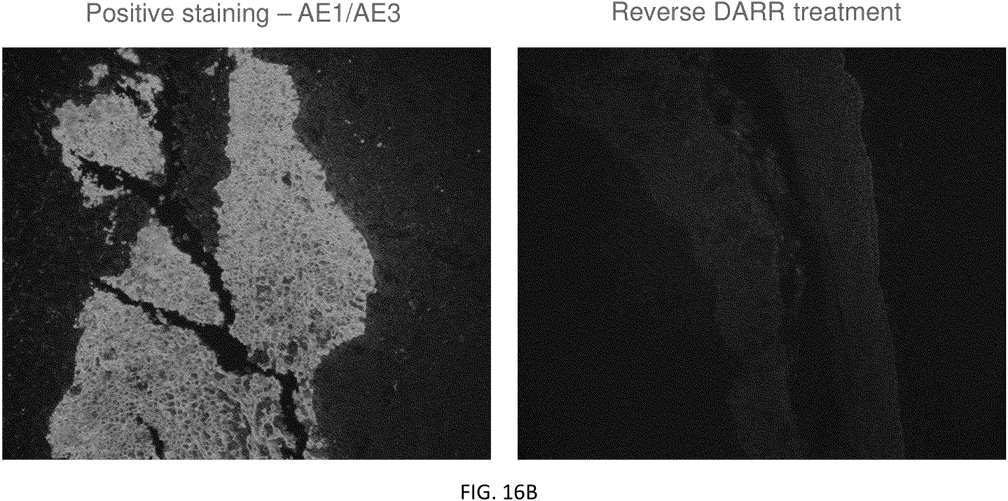

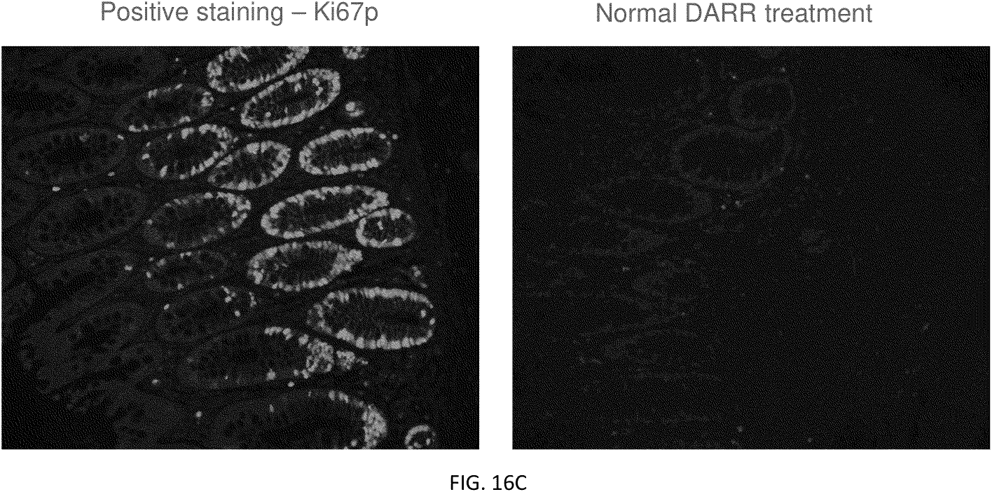

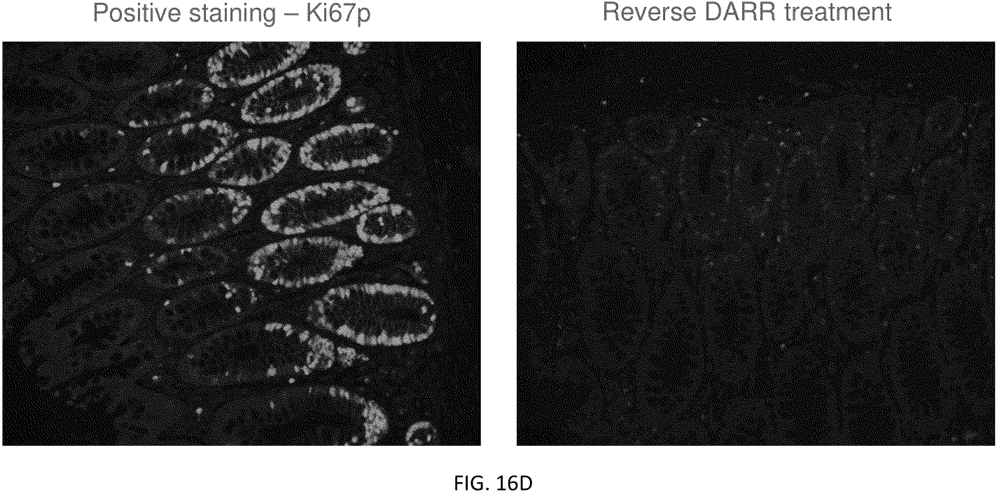

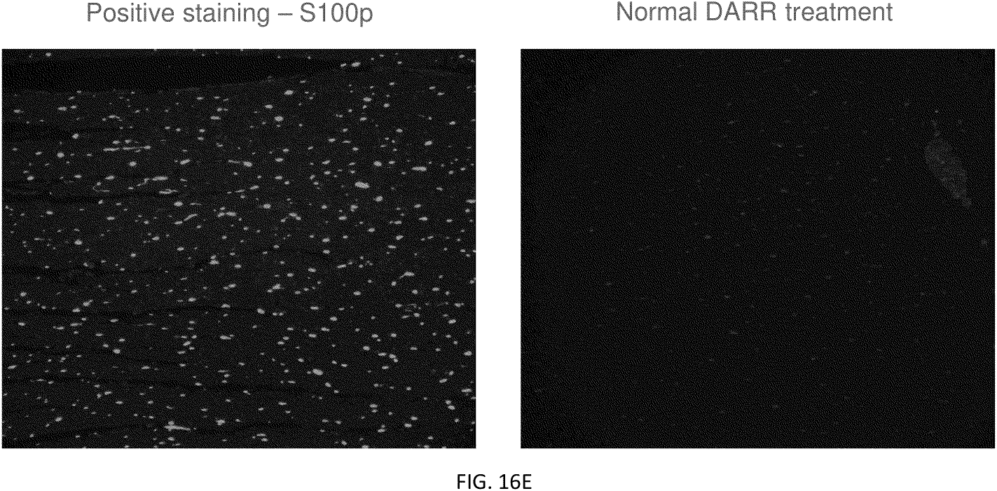

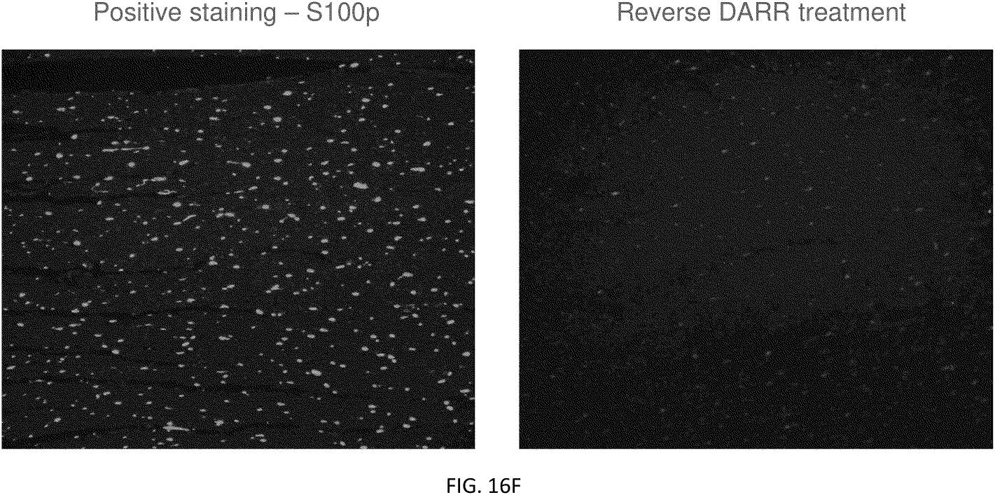

As shown in FIG. 16, each marker is substantially removed with DARR treatment according to the standard order. The performance is fair when Reagent 2 is used first. DARR is most effective when the reductant (Reagent 1) is applied first.

### Example 17

### DARR Treatment With Non-Tissue Surfaces

### Method

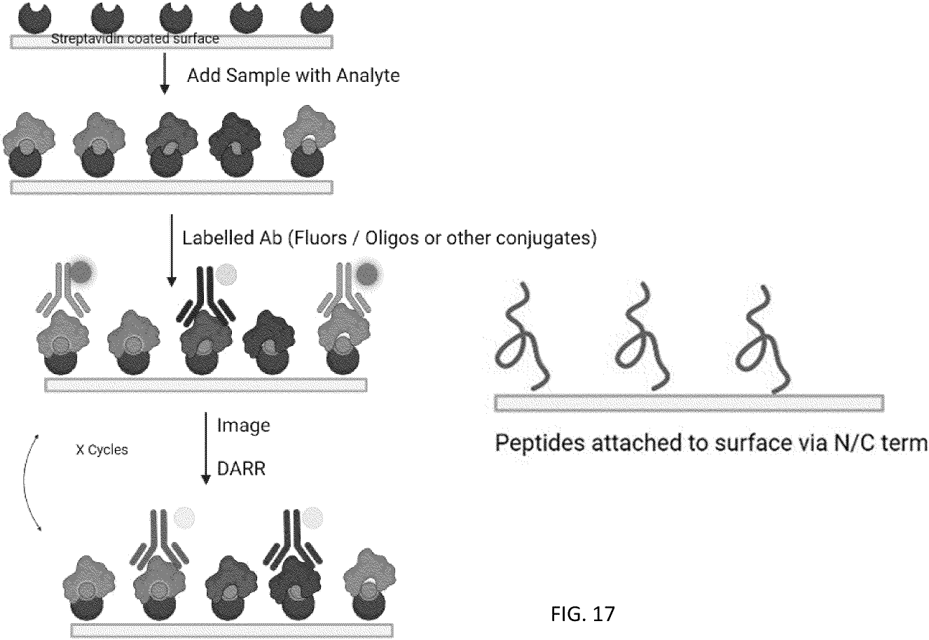

As shown in FIG. 17 Common surfaces used in flow cells or microfluidic cartridges include bare glass and Si wafers. Such surfaces can be coated with a range of different functional groups (amino silane, Streptavidin et). Flowcells can be used for detection of different analytes (proteins/ nucleic acids, etc.) through a single or sequential binding chemistry using labelled antibodies or other detection agents. Protocol steps for alternative surface treatment include:

1. The cell lysate, protein, or nucleic acid, or any analyte that can have a binding reagent made to bind it, is loaded onto the flow cell or cartridge in an appropriate buffer (analyte).

2. Incubate at an appropriate temp and for a certain amount of time.

3. Wash steps to remove unbound proteins.

4. Add labelled or conjugated antibodies.

5. Use DARR.

6. Repeat cycles.

Hydrogels can be grown on such surfaces. Hydrogels can be grown (polymerized) with analyte directly embedded, and amplification/detection assays can be performed directly in the growing gel. Labelled binding reagent can be leveraged for detection either during or after hydrogel formation. (See also U.S. Pat. No. 10,876,148B2 and U.S. Pat. Application No. 2022186310A1.)

For example, a surface is coated with streptavidin. A sample with an analyte is then added. A labeled antibody is then added, and images are subsequently obtained. A number of DARR stripping cycles are then performed, and one or more steps of the process are repeated. The process can use 4 or 2 color chemistry.

The analyte can be biotinylated DNA, DNA nanoballs, protein, peptides, etc. The analyte can also be a nucleic acid, protein (native or denatured), or other marker. Peptides can attach to the surface using the N or C termini. Several cycles need to be performed to detect the proteins/peptides. FIG. 17 depicts an exemplary workflow.

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those skilled in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

In the present disclosure, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

The practice of the present invention will employ, unless otherwise indicated, techniques of molecular biology, microbiology, cell biology, biochemistry, nucleic acid chemistry, and immunology. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

All publications mentioned herein are incorporated herein by reference for the purpose of describing and disclosing devices, compositions, formulations and methodologies which are described in the publication and which might be used in connection with the present disclosure.

The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Specific details are given in the present description to provide an understanding of the embodiments. However, it is understood that the embodiments may be practiced without these specific details.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included, and others specifically excluded in diverse embodiments.

Although the application has been disclosed in the context of certain embodiments and examples, it will be understood by those skilled in the art that the embodiments of the disclosure extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, now their specific configuration, communication and physical orientation with respect to each other. One skilled in the art should readily appreciate that these components can have various configurations and physical orientations (e.g., wholly separate components, units and subunits of groups of components, different communication regimes between components).

In closing, it is to be understood that the embodiments of the application disclosed herein are illustrative of the principles of the embodiments of the disclosure. Although specific embodiments and applications of the disclosure have been described in this specification, these embodiments and applications are exemplary only, and many variations are possible. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A composition for removing one or more bound detection agent from a stained biological sample or surface comprising:
(i) a reductant; and
(ii) a denaturant.

2. The composition of claim 1, wherein the biological sample comprises a tissue sample, or wherein the surface comprises a sensor, an electrode, and/or a hydrogel.

3. (canceled)

4. (canceled)

5. (canceled)

6. The composition of claim 1, wherein the reductant comprises at least one of beta-mercaptoethanol, Lithium aluminum hydride (LiAlH4), Red-Al (NaAlH2(OCH2CH2OCH3)2), Nascent (atomic) hydrogen, Hydrogen without or with a suitable catalyst e.g. a Lindlar catalyst, Sodium amalgam (Na(Hg)), Sodium-lead alloy (Na + Pb), Zinc amalgam (Zn(Hg)), Diborane Sodium borohydride (NaBH4), Compounds containing the Fe2+ ion, such as iron(II) sulfate, Compounds containing the Sn2+ ion such as tin(II) chloride, Sulfur dioxide, Sulfite compounds, Dithionates (Na2S206), Thiosulfates (Na2S2O3), Iodides (KI), Hydrogen peroxide (H2O2), Hydrazine (Wolff-Kishner reduction), Diisobutylaluminium hydride (DIBAL-H), Oxalic acid (C2H2O4), Formic acid (HCOOH), Ascorbic acid (C6H806), Reducing sugars, Phosphites, hypophosphites, and phosphorous acid, Dithiothreitol (DTT), Carbon monoxide (CO), Cyanides in hydrochemical metallurgical processes, Carbon (C), Tris-2-carboxyethylphosphine hydrochloride (TCEP), and/or tris-hydroxypropyl phosphine (THPP), and wherein the denaturant comprises at least one of guanidinium thiocyanate, ammonium thiocyanate, urea, guanidine, guanidium chloride, lithium perchlorate, sodium dodecyl sulfate, acetic acid, trichloroacetic acid, sulfosalicylic acid, sodium bicarbonate, Dodecaborate (B₁₂H₁₂)⁻², ethanol, formaldehyde, and/or glutaraldehyde.

7. The composition of claim 6, wherein the reductant comprises tris-hydroxypropyl phosphine, and/or wherein the denaturant comprises guanidinium thiocyanate.

8. (canceled)

9. (canceled)

10. The composition of claim 7, wherein the reductant comprises tris-hydroxypropyl phosphine, and the denaturant comprises guanidinium thiocyanate.

11. The composition of claim 10, wherein the reductant comprises THPP in aqueous solution at a concentration of between about 1 to 100 mg/mL, and the denaturant comprises guanidinium thiocyanate in aqueous solution at a concentration of between about 1 M to 6 M.

12. (canceled)

13. (canceled)

14. (canceled)

15. The composition of claim 1, wherein the one or more bound detection agent comprises at least one of a dye, antibody, nanobody, lectin, glycan-binding protein, recombinant protein, peptide, antibody fragment such as Fab, streptavidin, avidin, dendrimer, aptamer, enzyme such as horse radish peroxidase, alkaline phosphatase, or glucose oxidase, oligonucleotide, optionally labeled probe such as an oligonucleotide probe, single-stranded DNA probe, double-stranded DNA probe, branched DNA probe, and/or RNA probe, and/or any combination thereof.

16. A method for removing one or more bound detection agent from a stained biological sample or surface, the method comprising:
(i) providing a stained biological sample or surface;
(ii) applying a reductant to the sample; and
(iii) applying a denaturant to the sample.

17. The method of claim 16, wherein the biological sample comprises a tissue sample, or wherein the surface comprises a sensor, an electrode, and/or a hydrogel.

18. (canceled)

19. (canceled)

20. (canceled)

21. The method of claim 17, wherein the surface comprises a sensor is-functionalized with one or more binding agent and/or reactive group.

22. (canceled)

23. (canceled)

24. (canceled)

25. The method of claim 16, wherein the one or more bound detection agent comprises at least one of a dye, antibody, nanobody, lectin, glycan-binding protein, recombinant protein, peptide, antibody fragment such as Fab, streptavidin, avidin, dendrimer, aptamer, enzyme such as horse radish peroxidase, alkaline phosphatase, or glucose oxidase, oligonucleotide, optionally labeled probe such as an oligonucleotide probe, single-stranded DNA probe, double-stranded DNA probe, branched DNA probe, and/or RNA probe, and/or any combination thereof.

26. (canceled)

27. The method of claim 16, wherein the bound detection agent is labelled, and wherein the label comprises a dye, an enzyme, a mass tag, a nanoparticle, a quantum dot, or an oligonucleotide.

28. (canceled)

29. (canceled)

30. (canceled)

31. (canceled)

32. (canceled)

33. (canceled)

34. The method of claim 16, wherein the reductant comprises at least one of beta-mercaptoethanol, Lithium aluminum hydride (LiAlH4), Red-Al (NaAlH2(OCH2CH2OCH3)2), Nascent (atomic) hydrogen, Hydrogen without or with a suitable catalyst e.g. a Lindlar catalyst, Sodium amalgam (Na(Hg)), Sodium-lead alloy (Na + Pb), Zinc amalgam (Zn(Hg)), Diborane Sodium borohydride (NaBH4), Compounds containing the Fe2+ ion, such as iron(II) sulfate, Compounds containing the Sn2+ ion such as tin(II) chloride, Sulfur dioxide, Sulfite compounds, Dithionates (Na2S206), Thiosulfates (Na2S2O3), Iodides (KI), Hydrogen peroxide (H2O2), Hydrazine (Wolff-Kishner reduction), Diisobutylaluminium hydride (DIBAL-H), Oxalic acid (C2H2O4), Formic acid (HCOOH), Ascorbic acid (C6H806), Reducing sugars, Phosphites, hypophosphites, and phosphorous acid, Dithiothreitol (DTT), Carbon monoxide (CO), Cyanides in hydrochemical metallurgical processes, Carbon (C), Tris-2-carboxyethylphosphine hydrochloride (TCEP) and/or tris-hydroxypropyl phosphine (THPP), and/or wherein the denaturant comprises at least one of guanidinium thiocyanate, ammonium thiocynate, urea, guanidine, guanidium chloride, lithium perchlorate, sodium dodecyl sulfate, acetic acid, trichloroacetic acid, sulfosalicylic acid, sodium bicarbonate, Dodecaborate (B₁₂H₁₂)⁻², ethanol, formaldehyde, and/or glutaraldehyde.

35. The method of claim 34, wherein the reductant comprises tris-hydroxypropyl phosphine, and/or wherein the denaturant comprises guanidinium thiocyanate.

36. (canceled)

37. (canceled)

38. The method of claim 35, wherein the reductant comprises tris-hydroxypropyl phosphine, and the denaturant comprises guanidinium thiocyanate.

39. The method of claim 38, wherein the reductant comprises THPP in aqueous solution at a concentration of between about 1 to 100 mg/mL, and the denaturant comprises guanidinium thiocyanate in aqueous solution at a concentration of between about 1 M to 6 M.

40. (canceled)

41. (canceled)

42. (canceled)

43. The method of claim 16, further comprising one or more staining steps, and further comprising a second, third, fourth, fifth, sixth, or seventh detection agent removal step.

44. (canceled)

45. The method of claim 43, wherein the tissue integrity and/or morphology and/or nuclear features and/or antigenicity are preserved after the first, second, third, fourth, fifth, sixth, and/or seventh detection agent removal steps.

46. A sequential method for multiplex detection comprising:
(i) identifying a plurality of target markers of interest in a biological sample or a surface;
(ii) staining the biological sample or surface with a first plurality of detection agents specific for a first plurality of target markers of interest;
(iii) optionally, detecting a visual signal that indicates the presence or absence of the markers of interest in step (ii);
(iv) removing the first plurality of bound detection agents from the stained biological sample or surface;
(v) staining the biological sample or surface with a second plurality of detection agents specific for a second plurality of target markers of interest;
(vi) optionally, detecting a visual signal that indicates the presence or absence of the markers of interest of step (v); and
(vii) optionally, repeating steps (iv)-(vi) for detection of additional markers of interest.

47. (canceled)

48. The method of claim 46, wherein the biological sample comprises a tissue sample, or wherein the surface comprises a sensor, an electrode, and/or a hydrogel.

49. (canceled)

50. (canceled)

51. (canceled)

52. (canceled)

53. The method of claim 46, wherein steps (ii)-(vi) are repeated 1, 2, 3, 4, 5, 6, 7, 8, 9, or 10 times.

54. (canceled)

55. (canceled)

56. (canceled)

57. The method of claim 46, wherein the method for removing a bound detection agent from a stained biological sample comprises:
(i) applying a reductant to the sample; and
(ii) applying a denaturant to the sample.

58. The method of claim 46, wherein the reductant comprises at least one of beta-mercaptoethanol, Lithium aluminum hydride (LiAlH4), Red-Al (NaAlH2(OCH2CH2OCH3)2), Nascent (atomic) hydrogen, Hydrogen without or with a suitable catalyst e.g. a Lindlar catalyst, Sodium amalgam (Na(Hg)), Sodium-lead alloy (Na + Pb), Zinc amalgam (Zn(Hg)), Diborane Sodium borohydride (NaBH4), Compounds containing the Fe2+ ion, such as iron(II) sulfate, Compounds containing the Sn2+ ion such as tin(II) chloride, Sulfur dioxide, Sulfite compounds, Dithionates (Na2S2O6), Thiosulfates (Na2S2O3), Iodides (KI), Hydrogen peroxide (H2O2), Hydrazine (Wolff-Kishner reduction), Diisobutylaluminium hydride (DIBAL-H), Oxalic acid (C2H2O4), Formic acid (HCOOH), Ascorbic acid (C6H8O6), Reducing sugars, Phosphites, hypophosphites, and phosphorous acid, Dithiothreitol (DTT), Carbon monoxide (CO), Cyanides in hydrochemical metallurgical processes, Carbon (C), Tris-2-carboxyethylphosphine hydrochloride (TCEP) and/or tris-hydroxypropyl phosphine (THPP), and/or wherein the denaturant comprises at least one of guanidinium thiocyanate, ammonium thiocynate, urea, guanidine, guanidium chloride, lithium perchlorate, sodium dodecyl sulfate, acetic acid, trichloroacetic acid, sulfosalicylic acid, sodium bicarbonate, Dodecaborate (B₁₂H₁₂)⁻², ethanol, formaldehyde, and/or glutaraldehyde.

59. The method of claim 58, wherein the reductant comprises tris-hydroxypropyl phosphine, and/or wherein the denaturant comprises guanidinium thiocyanate.

60. (canceled)

61. (canceled)

62. The method of claim 59, wherein the reductant comprises tris-hydroxypropyl phosphine, and the denaturant comprises guanidinium thiocyanate.

63. (canceled)

64. (canceled)

65. The method of claim 46, wherein the process of detecting a visual signal comprises capturing an image of the stained biological sample or surface, and wherein the captured images from each sequential round of staining are compiled into a single multiplexed image.

66. (canceled)

67. A kit for removing a bound detection agent from a stained biological sample comprising the composition of claim 1.

68. (canceled)
